SEQUENCE LISTING (6 sequences)

SEQ ID NO: 1 - protein, 12 aa, synthetic construct
GSWKYWFGEGGC

SEQ ID NO: 2 - protein, 10 aa, synthetic construct
WRFWLKRGGC

SEQ ID NO: 3 - protein, 7 aa, synthetic construct
WRFWLKR

SEQ ID NO: 4 - protein, 8 aa, synthetic construct
WRFWLKRC

SEQ ID NO: 5 - protein, 10 aa, synthetic construct
CGGWRFWLKR

SEQ ID NO: 6 - protein, 8 aa, synthetic construct
CWRFWLKR

(19) United States
(12) Patent Application Publication
(10) Pub. No.: US 2026/0295070 A1
(43) Pub. Date: Oct. 1, 2026

(54) ADIPOSE TARGETING PEPTIDES, COMPOSITIONS, AND METHODS OF USE THEREOF

(71) Applicants: Shu Wang, Phoenix, AZ (US); Aysegul Kartal Acet, Phoenix, AZ (US); Fang Zhou, Tempe, AZ (US)

(72) Inventors: Shu Wang, Phoenix, AZ (US); Aysegul Kartal Acet, Phoenix, AZ (US); Fang Zhou, Tempe, AZ (US)

(73) Assignee: Arizona Board of Regents on behalf of Arizona State University, Scottsdale, AZ (US)

(21) Appl. No.: 19/630,819

(22) Filed: Mar. 27, 2026

Related U.S. Application Data
(60) Provisional application No. 63/778,791, filed on Mar. 27, 2025.

(51) Int. Cl. A61K 47/69; A61K 8/02; A61K 8/64; A61K 9/00; A61K 31/05; A61K 31/353; A61K 47/10; A61K 47/62; A61P 35/00; A61Q 19/08; C07K 7/06

(52) U.S. Cl. CPC A61K 47/6929; A61K 8/0241; A61K 8/64; A61K 9/0019; A61K 31/05; A61K 31/353; A61K 47/10; A61K 47/62; A61P 35/00; A61Q 19/08; C07K 7/06

(57) ABSTRACT

The present invention provides peptides that selectively target adipose tissue and adipose stromal stem cells (ASCs). Accordingly, in some embodiments, the invention provides compositions and methods for selective delivery of compounds to adipose tissue and ASCs and methods of treating diseases and disorders through targeted compound delivery.

### CROSS-REFERENCE TO RELATED APPLICATIONS

This application claims priority to U.S. Provisional Application No. 63/778,791 filed Mar. 27, 2025.

### REFERENCE TO A SEQUENCE LISTING SUBMITTED AS AN XML FILE

This application hereby incorporates by reference the entire contents of the Sequence Listing File named “206339-0130-00US_sequencelisting.xml” in XML format, with a creation date of Mar. 27, 2026, and a size of 6,108 bytes.

### BACKGROUND OF THE INVENTION

Prevalence of obesity and its related metabolic diseases is increasing in the U.S. and worldwide, and there is a critical need for developing an effective and safe therapeutic product. Pathologically expanded white adipose tissue (WAT) is strongly associated with increased multiple comorbidities, such as diabetes, cardiovascular disease, and certain types of cancer. Current lifestyle intervention (low compliance), pharmacotherapy (low efficacy), and bariatric surgery (high side effects and cost) have many disadvantages and cannot eradicate obesity in general populations. More practical and better approaches to combating obesity are urgently needed.

Adipose stromal stem cells (ASCs) are multipotent progenitor cells found in the stromal vascular fraction (SVF) of adipose tissue. ASCs play a critical role in adipose tissue maintenance, regeneration, and metabolic regulation. ASCs also play a role in the formation, progression, and relapse of cancer, wrinkle formation, and tissue regeneration.

Many therapeutic agents suffer from poor solubility, poor bioavailability, and high non-specific distribution in a subject which limit their application. Thus, there is a need in the art for compositions and methods for the targeted delivery of therapeutic agents to adipose tissue and ASCs. The present invention satisfies this unmet need.

### SUMMARY OF THE INVENTION

In some aspects, the present disclosure provides a composition comprising at least one targeting peptide comprising an amino acid sequence least 90% identical to SEQ ID NO:2, SEQ ID NO:3, SEQ ID NO:4, SEQ ID NO:5, or SEQ ID NO:6. In some embodiments, the composition comprises a nanoparticle comprising the at least one targeting peptide on or conjugated to at least a portion of the surface of the nanoparticle.

In some embodiments, the composition further comprises a therapeutic agent. In some embodiments, the therapeutic agent comprises an adipocyte stromal cell browning agent. In some embodiments, the therapeutic agent comprises at least one of the group consisting of: resveratrol, trans-resveratrol, and quercetin. In some embodiments, the composition comprises the at least one targeting peptide conjugated to the therapeutic agent directly or indirectly through a linker.

In some embodiments, the therapeutic agent is encapsulated within, adhered to a surface of, or integrated into the structure of a nanoparticle. In some embodiments, the composition increases bioavailability of resveratrol, or a derivative, prodrug, or pharmaceutically acceptable salt thereof, trans-resveratrol, or a derivative, prodrug, or pharmaceutically acceptable salt thereof, quercetin, or a derivative, prodrug, or pharmaceutically acceptable salt thereof, or any combination thereof.

In some embodiments, the composition induces browning of a white adipose tissue. In some embodiments, the composition induces the differentiation of an adipose stromal cell. In some embodiments, the differentiation comprises differentiation to a brown adipocyte or a beige adipocyte.

In some embodiments, the composition is selected from the group consisting of: an oral formulation, an injectable solution, a solution for transdermal delivery, an injectable hydrogel, a surgically implanted hydrogel, and any combination thereof.

In some embodiments, the nanoparticle of the composition comprises a poloxamer.

In some embodiments, the nanoparticle of the composition comprises poloxamer 188.

In some aspects, the present disclosure provides a method of treating a subject comprising the step of: administering a composition of the present invention.

In some embodiments, the subject has a metabolic disease, a metabolic disorder, or cancer.

In some embodiments, the subject has breast cancer.

In some embodiments, the treatment induces weight loss, reduces wrinkles, or promotes tissue regeneration.

In some embodiments, the subject has a metabolic disease or disorder selected from the group consisting of: obesity, obesity-related disease or disorder, diabetes, fatty liver disease, non-alcoholic fatty liver disease, cardiovascular disease, heart disease, stroke, hypertension, idiopathic intracranial hypertension, coronary artery disease, atrial fibrillation, atherosclerosis, venous thromboembolism, obstructive sleep apnea, obesity hypoventilation syndrome, metabolic syndrome, insulin resistance, esophageal adenocarcinoma, gastric cancer, colorectal cancer, hepatocellular carcinoma, cholangiocarcinoma, pancreatic cancer, endometrial carcinoma, ovarian cancer, breast cancer, renal cell carcinoma, multiple myeloma, and any combination thereof.

In some embodiments, the composition is administered via oral administration, injection, or a combination thereof.

### BRIEF DESCRIPTION OF THE DRAWINGS

The following detailed description of various embodiments of the invention are better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts representative fluorescence images of glycanation site-deficient decorin receptor (ΔDCN)-transduced 3T3-L1 (ΔDCN) cells after treatment with Rhoda-labeled non-targeted-R-NPs, ASC1-R-NPs, and ASC2-R-NPs for 2 hours at 37° C. The images are representative of three independent experiments.

FIG. 2 depicts DiIC₁₈ (7); 1,1′-dioctadecyl-3,3,3′,3′-tetramethylindotricarbocyanine iodide (DiR) fluorescence images of isolated inguinal white adipose tissue (I-WAT), upper subcutaneous adipose tissue (U-subc), and liver of C57BL/6J mice after treating them with DiR-labeled non-targeted-R-NPs, ASC1-R-NPs, and ASC2-R-NPs via tail vein. After 16 hours of injection, the mouse heart was perfused with 1× Phosphate-Buffered Saline (1×PBS), and In Vivo Imaging System (IVIS) was used to visualize the DiR accumulation in tissues and organs.

FIG. 3 depicts representative fluorescence images of ΔDCN-transduced 3T3-L1 cells (ΔDCN cells) after treatment with Rhoda-labeled non-targeted-R-NPs, ASC1-R-NPs, and ASC2-R-NPs.

FIG. 4 depicts DiIC₁₈ (7); 1,1′-dioctadecyl-3,3,3′,3′-tetramethylindotricarbocyanine iodide (DiR) fluorescence images of isolated inguinal whitef adipose tissue (I-WAT) of C57BL/6J mice after treating them with DiR-labeled non-targeted-R-NPs, ASC1-R-NPs, and ASC2-R-NPs via tail vein. In Vivo Imaging System (IVIS) was used to visualize the DiR accumulation.

FIG. 5 depicts DiIC₁₈ (7); 1,1′-dioctadecyl-3,3,3′,3′-tetramethylindotricarbocyanine iodide (DiR) fluorescence images of isolated upper subcutaneous adipose tissue (Upper Subc) of C57BL/6J mice after treating them with DiR-labeled non-targeted-R-NPs, ASC1-R-NPs, and ASC2-R-NPs via tail vein. After 16 hours of injection, the mouse heart was perfused with 1×PBS, and In Vivo Imaging System (IVIS) was used to visualize the DiR accumulation.

FIG. 6 depicts DiIC₁₈ (7); 1,1′-dioctadecyl-3,3,3′,3′-tetramethylindotricarbocyanine iodide (DiR) fluorescence images of isolated liver of C57BL/6J mice after treating them with DiR-labeled non-targeted-R-NPs, ASC1-R-NPs, and ASC2-R-NPs via tail vein. In Vivo Imaging System (IVIS) was used to visualize the DiR accumulation.

FIG. 7 depicts representative gene expression levels of browning markers in ΔDCN cells treated with R-NPs, ASC1-R-NPs, and ASC2-R-NPs measured using real time-qPCR.

FIG. 8 depicts representative images showing the results of a cell binding test.

### DETAILED DESCRIPTION

The present invention is based, in part, on the discovery of targeting peptides ASC1 and ASC2, which selectively target adipose tissue. In some embodiments, the targeting peptides of the invention can target adipose stromal cells (ASCs). The targeting peptides of the invention can bind to the glycanation site-deficient decorin (ΔDCN) receptor of ASCs. In some embodiments, the targeting peptides of the invention can target cells comprising a ΔDCN receptor. Accordingly, in some aspects, the present invention provides adipose-targeting peptides. ASC1 targeting peptide comprises the amino comprises the amino acid sequences of GSWKYWFGEGGC (SEQ ID NO:1). ASC2 targeting peptide comprises at least one of the amino acid sequences of WRFWLKRGGC (SEQ ID NO:2), WRFWLKR (SEQ ID NO:3), WRFWLKRC (SEQ ID NO:4), CGGWRFWLKR (SEQ ID NO:5), and CWRFWLKR (SEQ ID NO:6). In certain embodiments, ASC2 targeting peptide comprises the amino acid sequence of SEQ ID NO:3 in addition to any suitable nanoparticle linker sequence(s). Nanoparticle linker sequences may aid in linking the targeting peptide to a nanoparticle or any nanoparticle component. Nanoparticle linker sequences may aid in conjugation of the peptide to a second polypeptide. One or more nanoparticle linker sequences may be positioned at the N-terminal and/or C-terminal of SEQ ID NO:3. Exemplary nanoparticle linker sequences comprise the amino acid sequences of GGC, CGG, and C.

In another aspect, the targeting peptides of the present invention can be used for targeting of nanoparticles or compositions thereof to adipose tissue, an adipose tissue of interest, and/or ASCs. In some embodiments, a nanoparticle may comprise one or more targeting peptides of the invention. In some embodiments, the nanoparticle further comprises a therapeutic agent, a diagnostic agent, an imaging agent, or any combination thereof.

In some aspects, the present invention relates to methods of administration of a composition comprising one or more targeting peptides of the invention to a subject. In some aspects, the present invention relates to a method of treating and/or preventing a disease or disorder comprising administering to a subject a composition comprising one or more targeting peptides of the invention. Exemplary diseases or disorders comprise metabolic diseases or disorders, obesity, diabetes, cardiovascular disease, any obesity-related disease, and/or any type of cancer including breast cancer. In some aspects, the present invention relates to a method of reducing wrinkles or preventing wrinkle formation in a subject comprising administering to a subject a composition comprising one or more targeting peptides of the invention. In some aspects, the present invention relates to a method of regenerating tissue comprising administering to a subject a composition comprising one or more targeting peptides of the invention. In some aspects, the present invention relates to a method of browning or beigeing adipose tissue in a subject comprising administering to a subject a composition comprising one or more targeting peptides of the invention. In some aspects, the present invention relates to a method of browning or beigeing ASCs in a subject comprising administering to a subject a composition comprising one or more targeting peptides of the invention.

### Definitions

Unless defined otherwise, all technical and scientific terms used herein have the same meaning as commonly understood by one of ordinary skill in the art to which this invention belongs. Any methods and materials similar or equivalent to those described herein can be used in the practice or testing of the present invention.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, for example numerical values and/or ranges, such as an amount, a temporal duration, and the like, is meant to encompass variations of ±20%, ±10%, ±5%, ±1%, or ±0.1% from the specified value, as such variations are appropriate to perform the disclosed methods. For example, “about 40 [units]” may mean within ±25% of 40 (e.g., from 30 to 50), within ±20%, ±15%, ±10%, ±9%, ±8%, ±7%, ±6%, ±5%, ±4%, ±3%, ±2%, ±1%, less than ±1%, or any other value or range of values therein or therebelow. Furthermore, the phrases “less than about [a value]” or “greater than about [a value]” should be understood in view of the definition of the term “about” provided herein.

As used herein, the term “conjugate” means a product formed by covalent or non-covalent linkage of the targeting peptide (ASC1 and/or ASC2) that selectively targets an adipose tissue or ASC to an agent, such as a drug, lipid, polymer, peptide, polypeptide, polypeptide variant, etc.

As used herein, the term “nanoparticle” refers to particles having a particle size on the nanometer scale (e.g., about 1 nm-10,000 nm). For example, the nanoparticle may have a particle size up to about 2,000 nm. In another example, the nanoparticle may have a particle size up to about 100 nm. In another example, the nanoparticle may have a particle size up to about 6 nm. As used herein, “nanoparticle” refers to a number of nanoparticles, including, but not limited to, liposomes, lipid nanoparticles, polymer nanoparticles, organic nanoparticles, inorganic nanoparticles, biocompatible nanoparticles, such as biocompatible organic nanoparticles, biocompatible inorganic nanoparticles, etc., nanoclusters, nanocapsules, core-shell nanocapsules, nanovesicles, micelles, block copolymer micelles, lamaellae shaped particles, polymersomes, dendrimers, emulsions, exosomes, self-emulsifying drug delivery systems (SEDDS), microspheres, micro-structured lipid carriers, nano-structured lipid carriers, and other nano-size particles of various other small fabrications that are known to those of skill in the art. Examples of suitable nanoparticles useful in the invention include, but are not limited to, those described in Wang et al., 2014, J. Nutri. Biochem., 25:363-376, which is incorporated herein by reference in its entirety. The shapes and compositions of nanoparticles may be guided during condensation of atoms by selectively favoring growth of particular crystal facets to produce spheres, rods, wires, discs, cages, core-shell structures and many other shapes. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

As used herein, the phrase “lipid nanoparticle” refers to a transfer vehicle comprising at least one lipid (e.g., cationic lipids, non-cationic lipids, polar lipids, and PEG-modified lipids) and/or at least one polymers. Examples of suitable lipids include, but are not limited to, the phospholipid compounds (e.g., phosphatidylglycerol, phosphatidylcholine, phosphatidylserine, phosphatidylethanolamine, sphingolipids, cerebrosides, and gangliosides). Examples of suitable polymers include, but are not limited to, polyacrylates, polyalkycyanoacrylates, polylactide, polylactide-polyglycolide copolymers, polycaprolactones, dextran, albumin, gelatin, alginate, collagen, chitosan, cyclodextrins, dendrimers, and polyethylenimine.

The term “liposome” as used herein refers to microscopic vesicles or particles made up of at least one lipid bilayer enclosing an internal aqueous medium. To form liposomes, the presence of at least one “vesicle-forming lipid” is needed, which is an amphipathic lipid capable of either forming or being incorporated into a lipid bilayer. Any suitable vesicle-forming lipid may be used to form the lipid bilayer constituting the liposomes. Vesicle-forming lipid includes, but not limited to, phospholipids such as phosphatidylcholine (PC), phosphatidylglycerol (PG), phosphatidylinositol (PI), phosphatidic acid (PA), phosphatidylethanolamine (PE) or phosphatidylserine (PS), and charged lipids, such as a positively charge lipid or a negatively charged lipid. The term “liposome” may have the same meaning and may be interchanged with the term “lipid vesicle.” Liposome examples include large unilamellar vesicles, multilamellar vesicles, paucilamellar vesicles, small unilamellar vesicles, reverse phase evaporation vesicles, French press vesicles, and ether injection vesicles. Products incorporating liposomes include, but are not limited to, adjuvants, drug carriers, and cleansers. The following references disclose non-limiting examples of methods and/or non-limiting examples of apparatuses for manufacturing liposomes: “LIPOSOMES-Potential for Commercial Application”, by Dr. Norman D. Weiner, presented at the Emulsion-Suspension Technology Conference, Oct. 20-23, 1997, at New Brunswick, N.J.; U.S. Pat. No. 4,911,928 to Wallach, issued Mar. 27, 1990; U.S. Pat. No. 4,855,090 to Wallach, issued Aug. 8, 1989; and U.S. Pat. No. 4,895,452 to Yiournas et al., issued Jan. 23, 1990.

For example, the term “nanocapsule” refers to a vesicular system or hollow particle with a shell surrounding a core-forming space, which, in certain instances, can be used for transporting a payload on a nanoscale level. A nanocapsule may also be a nano-sized version of a container. The payload of the nanocapsule can be, but is not limited to drugs, medicaments, pharmaceutical compositions, chemical compositions, therapeutic compositions, biological macromolecules, dyes, biological material, immunological compositions, nutritional compositions, vitamins, proteins, nucleic acids, antibodies, and vaccines. Various materials may be used for producing such nanocapsules. Nanocapsule refers to a particle having a hollow core that is surrounded by a shell, such that the particle has a size of less than about 1000 nanometers. When a nanocapsule includes a bioactive component, the bioactive component is located in the core that is surrounded by the shell of the nanocapsule.

As used herein, the term “nanocage” refers to a nanocapsule, whereby the shell is not solid, as described for the nanocapsule, but has multiple holes or pores in its shell, thereby making it possible for the payload within the core of the nanocage to come into contact with the surrounding environment. These holes or pores may be regular or irregular in shape and/or spacing on the surface of the particle.

The term “micelle,” a useful article in the employment of a general aspect of the present invention, can generally be thought of as a small-on the order of usually nanometers in diameter-aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

The term “polymersome” as used herein refers to a vesicle-type which is typically composed of block copolymer amphiphiles, i.e., synthetic amphiphiles that have an amphiphilicity similar to that of lipids. By virtue of their amphiphilic nature (having a more hydrophilic block (head) and a more hydrophobic block (tail)), the block copolymers are capable of self-assembling into a head-to-tail and tail-to-head bilayer structure similar to liposomes. Compared to liposomes, polymersomes have much larger molecular weights, with number average molecular weights typically ranging from 1,000 to 100,000, from 2,500 to 50,000 or from 5,000 to 25,000, are typically chemically more stable, less leaky, less prone to interfere with biological membranes, and less dynamic due to a lower critical aggregation concentration. These properties result in less opsonisation and longer circulation times. The terms “more hydrophilic” and “more hydrophobic” as used in the context of the ampohiphilic nature of the block copolymers are used in a relative sense. i.e., both can be either hydrophilic or hydrophobic, as long as the difference in polarity between the blocks is sufficient for the formation of polymersomes according to the present invention. In view of the creation of a cavity in which water may be incorporated, in certain aspects the more hydrophilic end of the polymer is to be hydrophilic per se. Further, in view of the use as a therapeutic agent carrier, it is desired that also hydrophobic and/or hydrophilic therapeutic agents can be incorporated into the polymersomes. In some embodiments, the hydrophobic end of the polymer is hydrophobic per se. In some embodiments, the amphiphilic nature of the block copolymers is realized in the form of a block copolymer comprising a block made up of more hydrophilic monomeric units (A) and a block made up of more hydrophobic units (B), the block copolymer having the general structure AnBm, with n and m being integers of from 5 to 5,000, 10 to 1,000, or 10 to 500. It is also conceivable that at least one further units or blocks are built-in, e.g., a unit C with an intermediate hydrophilicity so as to yield a terpolymer having the general structure AnCpBm, with n and m being as defined above, and p being an integer of from 5 to 5,000, 10 to 1,000, or 10 to 500. Any of the blocks can itself be a copolymer, i.e., comprise different monomeric units of the required hydrophilic respectively hydrophobic nature. In some embodiments, the blocks themselves are homopolymeric. Any of the blocks, in particular the more hydrophilic block, may bear charges. The number and type of charges may depend on the pH of the environment. Any combination of positive and/or negative charges on any of the blocks is contemplated by the present invention.

“Dendrimers” have descriptions, definitions and understandings in the literature. For example, and without limitation and including other and similar definitions, descriptions and understandings in the art, the term dendrimer from the Greek word, “dendron,” for tree, can refer to a synthetic, three-dimensional molecule with branching parts. Descriptions and understandings of dendrimers can be gleaned from Holister et al., (Dendrimers, Technology White Papers nr. 6, pub. October 2003, cientifica), as well as the other literature published by those skilled in the art on dendrimers, all of which are incorporated herein by reference.

“Lamella” is a term whose definitions, descriptions and understandings are also known to those of skill in the art and which are incorporated herein by reference. In a very general sense, lamella or lamellae refers to plate-like, gill-shaped or other layered structures.

The definitions, descriptions and understandings of “nanovesicle” are well known to those of skill in the art, and are incorporated herein by reference. For example, “nanovesicle” can refer to a variety of small sac, sac-like or globular structures capable of containing fluid or other material therein.

As used herein, the term “exosome” refers to a subset of circulating microvesicles that are preformed microvesicles that are released from the cell following the exocytic fusion of intracellular multivesicular bodies with the plasma membrane, i.e., exosomes have an endocytic origin. As used herein, it is not intended that an exosome of the invention be limited by any particular size or size range. For example, exosomes include a variety of nanoparticles, including microvesicles, epididimosomes, argosomes, exosome-like vesicles, microparticles, promininosomes, prostasomes, dexosomes, texosomes, dex, tex, archeosomes and oncosomes. Exosomes are secreted by a wide range of cells, such as mammalian cells, and are secreted under both normal and pathological conditions. Exosomes, in some embodiments, function as intracellular messengers by virtue of carrying mRNA or other contents from a first cell to another cell (or plurality of cells). In some embodiments, exosomes are involved in blood coagulation, immune modulation, metabolic regulation, cell division, and other cellular processes.

The term “emulsion” as used herein refers to a mixture of two or more substances, such as liquids, which are normally immiscible, in which one substance forms droplets that are dispersed within another substance. One substance (the dispersed phase) is dispersed in the other (the continuous phase). Depending on the substances used, the droplets of an emulsion may be in the range of 1 nm to 100 μm, e.g., 1 nm to 100 nm, 1 μm to 50 μm, etc. For example, In some embodiments, the continuous phase is an aqueous phase, and the dispersed phase is an organic (oily or hydrophobic) phase; that is, the emulsion is an oil-in-water emulsion.

As used herein, the term “self-emulsifying drug delivery systems (SEDDS)” refers to isotropic mixtures, consisting of oils, surfactants, and/or cosolvents. In some embodiments, SEDDS increase solubility and bioavailability of poorly soluble drugs. For example, in some embodiments, designed SEDDS formulations are used to improve the oral absorption of highly lipophilic compounds. Examples of suitable SEDDS useful in the invention include, but are not limited to, those described in WO 2002/007712 A2 and Wang et al., 2014, J. Nutri. Biochem., 25:363-376, which are incorporated herein by reference in their entireties.

As used herein, the term “polymorph” refers to crystalline forms having the same chemical composition but different spatial arrangements of the molecules, atoms, and/or ions forming the crystal.

As used herein, the term “analog,” “analogue,” or “derivative” is meant to refer to a chemical compound or molecule made from a parent compound or molecule by at least one chemical reaction. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through at least one metabolic process. In some embodiments, upon in vivo administration, a prodrug is chemically converted to the biologically, pharmaceutically, or therapeutically active form of the compound. In another embodiment, a prodrug is enzymatically metabolized by at least one step or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid, and any combinations or derivatives thereof. Other examples include, but are not limited to, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya, and biosynthetic gum. Other examples of useful primary particle and/or secondary stabilizers include polymers, such as polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(amides), poly(vinylpyrrolidone).

The terms “coat,” “coated,” or “coating,” as used herein, refer to at least a partial coating of the nanoparticle. One hundred percent coverage is not necessarily implied by these terms.

An “effective amount” or “therapeutically effective amount,” as used herein, means an amount, which provides a therapeutic or prophylactic benefit. For example, an “effective amount” or “therapeutically effective amount” of a nanoparticle is that amount of compound, which is sufficient to provide a beneficial effect (e.g., deliver a nutritional or bioactive agent) to the subject to which the nanoparticle is administered.

“Pharmaceutically acceptable” refers to those properties and/or substances, which are acceptable to the subject from a pharmacological/toxicological point of view and to the manufacturing pharmaceutical chemist from a physical/chemical point of view regarding composition, formulation, stability, subject acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, anti-bacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art.

The term “pharmaceutically acceptable salt” refers to any pharmaceutically acceptable salt, which upon administration to the subject is capable of providing (directly or indirectly) a compound as described herein. In some embodiments, such salts are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts, such as, for example, acetate, trifluoroacetate, maleate, fumarate, citrate, oxalate, succinate, tartrate, malate, mandelate, methane sulphonate, and p-toluenesulphonate. Examples of the alkali addition salts include inorganic salts, such as, for example, sodium, potassium, calcium and ammonium salts, and organic alkali salts, such as, for example, ethylenediamine, ethanolamine, N,N-dialkylenethanolamine, triethanolamine, and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which the said compound is bonded by a non-covalent bond to another molecule (normally a polar solvent), including especially hydrates and alcoholates.

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration. The pharmaceutical composition facilitates administration of the compound to an organism. Multiple techniques of administering a compound exist in the art including, but not limited to, intravenous, oral, aerosol, parenteral, ophthalmic, pulmonary and topical administration.

The term “therapeutic” as used herein means a treatment and/or prophylaxis. A therapeutic effect is obtained by suppression, diminution, remission, or eradication of at least one sign or symptom of a disease or disorder state.

As used herein, the terms “therapeutic compound,” “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, hydrophilic therapeutic agents, hydrophobic therapeutic agents, antibiotics, antibodies, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, anti-hypercalcemia agents, mast cell stabilizers, muscle relaxants, nutritional or bioactive agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the at least one therapeutic agents is water-soluble, poorly water-soluble drug or a drug with a low, medium, or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the nanocluster. The active agent-nanocluster combination may be coated further to encapsulate the agent-nanocluster combination and may be directed to a target by functionalizing the nanocluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional or bioactive agents, and the like. The active agents described herein may be found in their native state, however, they will generally be provided in the form of a salt. The active agents described herein include their isomers, analogs, and derivatives.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

To “treat” a disease as the term is used herein, means to reduce the frequency or severity of at least one sign or symptom of a disease or disorder experienced by a subject.

As used herein, “treating a disease or disorder” means reducing the frequency with which a symptom of the disease or disorder is experienced by a patient. Disease and disorder are used interchangeably herein.

A disease or disorder is “alleviated” if the severity of a sign or symptom of the disease or disorder, the frequency with which such a sign or symptom is experienced by a patient, or both, is reduced.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject.

“Parenteral” administration of a composition includes, e.g., subcutaneous (s.c.), intravenous (i.v.), intramuscular (i.m.), or intrasternal injection, or infusion techniques.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the terms “amino acid”, “amino acidic monomer”, or “amino acid residue” refer to any of the twenty naturally occurring amino acids including synthetic amino acids with unnatural side chains and including both D and L optical isomers.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the discovery of targeting peptides that selectively target adipose tissue. In some embodiments, the adipose tissue is a specific adipose tissue of interest. In some embodiments, the targeting peptides selectively target adipose stromal cells (ASCs). In some embodiments, the targeting peptides of the invention selectively target or bind to the ΔDCN receptor of ASCs.

In some aspects, the present invention provides nanoparticles or compositions thereof comprising one or more of the targeting peptides of the invention. Nanoparticles or compositions thereof comprising one or more of the targeting peptides of ASC1 and ASC2 may deliver a therapeutic agent to ASCs. The therapeutic agent of the nanoparticles or compositions thereof of the invention may comprise any molecule that promotes a desired ASC phenotype when taken up by ASCs. In some embodiments, the therapeutic agent comprises a diagnostic agent and/or an imaging agent.

In some aspects, the present invention relates to a method for in targeted delivery of a therapeutic agent, or any combination thereof to adipose tissue or ASCs using at least one nanoparticle comprising one or more targeting peptides of the invention. In some embodiments, the present invention provides methods comprising administering to a subject a targeted nanoparticle or composition of the present invention. In some embodiments, the subject is a subject in need thereof.

In some embodiments, the present invention relates to a method of preventing or treating a metabolic disease or disorder or cancer. In some aspects, the present invention provides methods of reducing body fat, inducing weight loss, and/or improving glucose homeostasis and insulin sensitivity. In some aspects, the present invention relates to a method of preventing wrinkle formation or reducing wrinkles in a subject using at least one nanoparticle comprising one or more targeting peptides of the invention. In some aspects, the present invention relates to a method of promoting tissue regeneration in a subject using at least one nanoparticle comprising one or more targeting peptides of the invention.

In some aspects, the present invention provides methods of activating brown adipose tissue of interest. In other aspects, the present invention provides methods of browning or beigeing white adipose tissue of interest. In various aspects, the present invention also provides methods of differentiating ASCs to brown adipocytes or beige adipocytes. In various aspects, the present invention also provides methods of promoting a desired ASC phenotype or desired ASC differentiation outcome in a subject.

### Targeting Peptides

In various embodiments, the present invention provides targeting agents. In some embodiments, the targeting agents are targeting peptides. In some embodiments, the targeting peptides comprise ASC1 or ASC2, a peptide homologous to ASC1 or ASC2, or a derivative or mimetic thereof. In some embodiments, the targeting peptide comprises an amino acid sequence at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical to SEQ ID NOs: 1, 2, 3, 4, 5, or 6. In some embodiments, the targeting peptide comprises an amino acid sequence substantially homologous to SEQ ID NOs: 1, 2, 3, 4, 5, or 6. In some embodiments, the targeting peptide comprises an amino acid sequence that is a variant of SEQ ID NOs: 1, 2, 3, 4, 5, or 6. In some embodiments, the targeting peptide comprises an amino acid sequence substantially homologous to SEQ ID NOs: 1, 2, 3, 4, 5, or 6. In some embodiments, the targeting peptide comprises the amino acid sequence of SEQ ID NOs: 1, 2, 3, 4, 5, or 6. In some embodiments, the targeting peptide comprises the amino acid sequence of SEQ ID NO:2, 3, 4, 5, or 6. In some embodiments, the targeting peptide comprises the amino acid sequence of SEQ ID NO:3 and one or more nanoparticle linker sequence positioned at the N-terminal and/or C-Terminal of the amino acid sequence of SEQ ID NO:3.

As known in the art the “similarity” between two peptides is determined by comparing the amino acid sequence and its conserved amino acid substitutes of one peptide to a sequence of a second peptide. Variants are defined to include peptide sequences different from the original sequence, for example different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 85%, 90%, 91%, 92%, 93%, 94%, 95%, 96%, 97%, 98% or 99% similar or identical to the original amino acid sequence. The degree of identity between two peptides is determined using computer algorithms and methods that are widely known for the persons skilled in the art. In some aspects, the identity between two amino acid sequences is determined by using the BLASTP algorithm.

As used herein, an amino acid sequence is “substantially homologous” to any of the amino acid sequences described herein when its amino acid sequence has a degree of identity with respect to the amino acid sequence of at least 60%, at least 70%, at least 75%, at least 80%, at least 85%, at least 90%, or at least 95%. In some aspects, the identity between two amino acid sequences is determined by using the BLASTP algorithm.

In other embodiments, variant antibodies, antigen-binding fragment or bispecific antibodies comprising one or more conservative mutations in the antigen-binding domain are disclosed herein. The conservative mutations may reside in the well-known framework regions or any of the CDRs, as long as the variant antibodies, antigen-binding fragments retain the desired functional properties of the parent molecules, such as those described herein.

“Conservative modifications” refer to amino acid modifications that do not significantly affect or alter the binding characteristics of the antibody or an antigen-binding fragment containing the amino acid modifications. Conservative modifications include amino acid substitutions, insertions and deletions. Conservative amino acid substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., Acta Physiol. Scand. Suppl. 1988; 643:55-67; Sasaki et al., Adv. Biophys. 1988; 35:1-24).

TABLE 1
Conservative Amino Acid Substitutions

| | | 3 Letter | 1 Letter |
|---|---|---|---|
| Group | Amino Acid | Abbreviation | Abbreviation |
| Aromatic | Phenylalanine | Phe | F |
| | Tryptophan | Trp | W |
| | Tyrosine | Tyr | Y |
| Negatively Charged | Aspartate | Asp | D |
| | Glutamate | Glu | E |
| Non-Polar Aliphatic | Alanine | Ala | A |
| | Glycine | Gly | G |
| | Isoleucine | Ile | I |
| | Leucine | Leu | L |
| | Methionine | Met | M |
| | Valine | Val | V |
| Polar Uncharged | Asparagine | Asn | N |
| | Cysteine | Cys | C |
| | Glutamine | Gln | Q |
| | Proline | Pro | P |
| | Serine | Ser | S |
| | Threonine | Thr | T |
| Positively Charged | Arginine | Arg | R |
| | Histidine | His | H |
| | Lysine | Lys | K |

Within a group of amino acids, some substitutions may be preferred over others. For example, glycine and alanine may preferably be used to substitute for one another (since they have relatively short side chains) and valine, leucine and isoleucine may be used to substitute for one another (since they have larger aliphatic side chains which are hydrophobic). Amino acid substitutions to the antibodies described herein may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

In some embodiments, the peptides disclosed herein can be modified according to the methods known in the art for producing peptidomimetics, see, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746 (2003). In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N terminus to the C terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, an artificial amino acid analog. Artificial amino acid analogs include beta-amino acids, beta-substituted beta-amino acids (“beta3-amino acids”), phosphorous analogs of amino acids, such as b-amino phosphonic acids and b-amino phosphinic acids, and amino acids having non-peptide linkages. Artificial amino acids can be used to create peptidontimetics, such as peptoid oligomers (e.g., peptoid amide or ester analogues), beta-peptides, cyclic peptides, oligourea or oligocarbamate peptides; or heterocyclic ring molecules.

### Nanoparticles

The present invention relates, in part, to nanoparticles comprising one or more targeting agents of the present invention. In various embodiments, the nanoparticle selectively binds to or targets an adipose tissue, a specific adipose tissue of interest, ASCs, and/or the ΔDCN receptor of ASCs. In various embodiments, the one or more targeting agents are encapsulated within the nanoparticle, adhered to the surface of the nanoparticle, integrated into the structure of the nanoparticle, bound to the nanoparticle, or any combination thereof.

Exemplary nanoparticles for use with the targeting peptides of the invention are described in International Application No. PCT/US2024/038805 which is incorporated by reference herein in its entirety. Exemplary therapeutic agents for use with nanoparticles comprising the targeting agents of the invention and compositions thereof are also described in International Application No. PCT/US2024/038805.

In various embodiments, the nanoparticle comprises the one or more targeting agents in a concentration range of about 0.01 mol % to about 99.99 mol %. In some embodiments, the nanoparticle comprises the one or more targeting agents in a concentration range of about 0.1 mol % to about 99.9 mol %. In some embodiments, the nanoparticle comprises the one or more targeting agents in a concentration range of about 1 mol % to about 70 mol %. In some embodiments, the nanoparticle comprises the one or more targeting agents in a concentration range of about 1 mol % to about 60 mol %. In some embodiments, the nanoparticle comprises the one or more targeting agents in a concentration range of about 1 mol % to about 55 mol %. In some embodiments, the nanoparticle comprises the one or more targeting agents in a concentration range of about 1 mol % to about 50 mol %. In some embodiments, the nanoparticle comprises the one or more targeting agents in a concentration range of about 10 mol % to about 50 mol %.

For example, in some embodiments, the nanoparticle comprises one or more targeting agents in a concentration of about 0.01 mol %. In some embodiments, the nanoparticle comprises the one or more targeting agents in a concentration of about 0.02 mol %. In some embodiments, the nanoparticle comprises the one or more targeting agents in a concentration of about 0.05 mol %. In some embodiments, the nanoparticle comprises the one or more targeting agents in a concentration of about 0.15 mol %. In some embodiments, the nanoparticle comprises the one or more targeting agents in a concentration of about 1.0 mol %. In some embodiments, the nanoparticle comprises the one or more targeting agents in a concentration of about 10 mol %.

In some embodiments, the nanoparticle selectively targets at least one cell comprising a ΔDCN receptor. In some embodiments, the at least one cell is an ASC.

In various embodiments, the nanoparticle selectively targets the ΔDCN receptor. In some embodiments, the nanoparticle selectively binds the ΔDCN receptor. In some embodiments, the nanoparticle inhibits or reduces the activity of the ΔDCN receptor. In some embodiments, the nanoparticle promotes or increases the activity of the ΔDCN receptor. In various embodiments, the nanoparticles described herein are formulated for stability for in vivo cell targeting. In some embodiments, the nanoparticle formulated for stability for in vivo delivery to a cell of interest (e.g., a cell comprising a ΔDCN receptor or a fragment thereof, ASCs, etc.). In some embodiments, the ΔDCN receptor is involved in the uptake of nanoparticles. In some embodiments, the ΔDCN receptor is inhibited so that nanoparticle transcytosis is enhanced.

In various embodiments, the nanoparticle has an average hydrodynamic diameter of from about 10 nm to about 10,000 nm, from about 130 nm to about 2,500 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 100 nm, 200 nm, 300 nm, 400 nm, 500 nm, 600 nm, 700 nm, 800 nm, 900 nm, 1,000 nm, 2,000 nm, 2,500 nm, 5,000 nm, or 10,000 nm.

For example, in various embodiments, the nanoparticle has an average particle size (e.g., average hydrodynamic diameter of the nanoparticle) below about 2,500 nm. In some embodiments, the nanoparticle has a particle size (e.g., average hydrodynamic diameter of the nanoparticle) of between about 1 nm to about 2,500 nm. In some embodiments, the nanoparticle has a particle size (e.g., average hydrodynamic diameter of the nanoparticle) of between about 1 nm to about 300 nm. In some embodiments, the nanoparticle has a particle size (e.g., average hydrodynamic diameter of the nanoparticle) of between about 1 nm to about 200 nm. In some embodiments, the nanoparticle has a particle size (e.g., average hydrodynamic diameter of the nanoparticle) of below about 110 nm.

In some embodiments, the nanoparticle has a narrow size distribution. In some embodiments, the nanoparticles have a polydispersity index (PDI) of less than about 0.4, less than about 0.35, less than about 0.3, less than about 0.25, less than about 0.225, less than about 0.2, less than about 0.1. In some embodiments, the nanoparticles have a PDI between about 0.1 and about 0.01, between about 0.09 and about 0.02, between about 0.08 and about 0.03, between about 0.07 and about 0.04, between about 0.06 and about 0.05.

In some embodiments, the nanoparticle is any type of nanoparticle, including, but not limited to, liposomes, lipid nanoparticles, organic nanoparticles, inorganic nanoparticles (e.g., metal nanoparticles, such as gold nanoparticles, iron nanoparticles, ZnO nanoparticles, TiO₂ nanoparticles, etc.), biocompatible nanoparticles, such as biocompatible organic nanoparticles, biocompatible inorganic nanoparticles, etc., polymer nanoparticles, nanoclusters, nanocapsules, core-shell nanocapsules, nanovesicles, micelles, block copolymer micelles, lamaellae shaped particles, polymersomes, dendrimers, emulsions, exosomes, SEDDS, microspheres, micro-structured lipid carriers, nano-structured lipid carriers, and other nano-size particles of various other small fabrications that are known to those of skill in the art.

In various embodiments, the nanoparticle comprises at least one therapeutic agent (e.g., a therapeutic agent, diagnostic agent, browning agent, etc.). In some embodiments, the nanoparticle encapsulates at least one therapeutic agent. Thus, In some embodiments, the nanoparticle is a nanocapsule. In some embodiments, the nanoparticle is a nanocarrier.

### Lipid Nanoparticles

In some embodiments, the nanoparticle is a liposome. In some embodiments, the liposome comprises phospholipids, cholesterol, sphingolipids, ceramides, hapten-conjugated lipids, or any combination thereof.

In some embodiments, the nanoparticle is a lipid nanoparticle. In some embodiments, the lipid nanoparticles comprise phospholipids, cholesterol, PEG-phospholipids, lipid-soluble vitamins, lipid conjugates, or any combination thereof.

In some embodiments, the lipid is selected from a fatty acid, wax, sterol, lipid-soluble vitamin, such as vitamins A, D, E, and K, monoglyceride, diglyceride, triglyceride, phospholipids, cholesterol, PEG-phospholipids, lipid conjugates, or any combination thereof. In some embodiments, the phospholipid is selected from phosphatidylcholine (PC), such as soy or egg L-α-phosphatidylcholine (PC), phosphatidic acid (PA), phosphatidylethanolamine (cephalin) (PE), lecithin, phosphatidylserine (PS), phosphoinositide, phosphatidylinositol (PI), phosphatidylinositol phosphate (PIP), phosphatidylinositol bisphosphate (PIP2), phosphatidylinositol trisphosphate (PIP3), phosphosphingolipid, phospholipid-phosphatic acid, sphingolipid, ceramide phosphorylcholine (sphingomyelin) (SPH), ceramide phosphorylethanolamine (sphingomyelin) (Cer-PE), ceramide phosphoryl lipid, LysoPC, LysoPE, LysoPS, LysoPI, LysoPA, acyl-carnitine (e.g., C2-C28 acyl-carnitine), or any combination thereof.

In various embodiments, the nanoparticle comprises one or more lipids in a concentration range of about 0.1 mol % to about 100 mol %. In some embodiments, the nanoparticle comprises one or more lipids in a concentration range of about 1 mol % to about 100 mol %. In some embodiments, the nanoparticle comprises one or more lipids in a concentration range of about 1 mol % to about 70 mol %. In some embodiments, the nanoparticle comprises one or more lipids in a concentration range of about 10 mol % to about 80 mol %. In some embodiments, the nanoparticle comprises one or more lipids in a concentration range of about 10 mol % to about 70 mol %. In some embodiments, the nanoparticle comprises one or more lipids in a concentration range of about 10 mol % to about 50 mol %. In some embodiments, the nanoparticle comprises one or more lipids in a concentration range of about 15 mol % to about 45 mol %. In some embodiments, the nanoparticle comprises one or more lipids in a concentration range of about 35 mol % to about 40 mol %.

For example, in some embodiments, the nanoparticle comprises one or more lipids in a concentration of about 1 mol %. In some embodiments, the nanoparticle comprises one or more lipids in a concentration of about 2 mol %. In some embodiments, the nanoparticle comprises one or more lipids in a concentration of about 5 mol %. In some embodiments, the nanoparticle comprises one or more lipids in a concentration of about 5.5 mol %. In some embodiments, the nanoparticle comprises one or more lipids in a concentration of about 10 mol %. In some embodiments, the nanoparticle comprises one or more lipids in a concentration of about 12 mol %. In some embodiments, the nanoparticle comprises one or more lipids in a concentration of about 15 mol %. In some embodiments, the nanoparticle comprises one or more lipids in a concentration of about 20 mol %. In some embodiments, the nanoparticle comprises one or more lipids in a concentration of about 25 mol %. In some embodiments, the nanoparticle comprises one or more lipids in a concentration of about 30 mol %. In some embodiments, the nanoparticle comprises one or more lipids in a concentration of about 35 mol %. In some embodiments, the nanoparticle comprises one or more lipids in a concentration of about 37 mol %. In some embodiments, the nanoparticle comprises one or more lipids in a concentration of about 40 mol %. In some embodiments, the nanoparticle comprises one or more lipids in a concentration of about 45 mol %. In some embodiments, the nanoparticle comprises one or more lipids in a concentration of about 50 mol %. In some embodiments, the nanoparticle comprises one or more lipids in a concentration of about 60 mol %. In some embodiments, the nanoparticle comprises one or more lipids in a concentration of about 70 mol %. In some embodiments, the nanoparticle comprises one or more lipids in a concentration of about 80 mol %. In some embodiments, the nanoparticle comprises one or more lipids in a concentration of about 90 mol %. In some embodiments, the nanoparticle comprises one or more lipids in a concentration of about 95 mol %. In some embodiments, the nanoparticle comprises one or more lipids in a concentration of about 95.5 mol %. In some embodiments, the nanoparticle comprises one or more lipids in a concentration of about 99 mol %. In some embodiments, the nanoparticle comprises one or more lipids in a concentration of about 99.9 mol %. In some embodiments, the nanoparticle comprises one or more lipids in a concentration of about 100 mol %.

In various embodiments, the nanoparticle comprises at least one lipid in a concentration range of about 0.1 wt % to about 100 wt %. In some embodiments, the nanoparticle or composition comprises at least one lipid in a concentration range of about 1 wt % to about 100 wt %. In some embodiments, the nanoparticle or composition comprises at least one lipid in a concentration range of about 5 wt % to about 90 wt %. In some embodiments, the nanoparticle or composition comprises at least one lipid in a concentration range of about 1 wt % to about 70 wt %. In some embodiments, the nanoparticle or composition comprises at least one lipid in a concentration range of about 10 wt % to about 80 wt %. In some embodiments, the nanoparticle or composition comprises at least one lipid in a concentration range of about 10 wt % to about 70 wt %. In some embodiments, the nanoparticle or composition comprises at least one lipid in a concentration range of about 10 wt % to about 50 wt %. In some embodiments, the nanoparticle or composition comprises at least one lipid in a concentration range of about 15 wt % to about 45 wt %. In some embodiments, the nanoparticle or composition comprises at least one lipid in a concentration range of about 35 wt % to about 40 wt %.

For example, in some embodiments, the nanoparticle or composition comprises at least one lipid in a concentration of about 1 wt %. In some embodiments, the nanoparticle or composition comprises at least one lipid in a concentration of about 2 wt %. In some embodiments, the nanoparticle or composition comprises at least one lipid in a concentration of about 5 wt %. In some embodiments, the nanoparticle or composition comprises at least one lipid in a concentration of about 5.5 wt %. In some embodiments, the nanoparticle or composition comprises at least one lipid in a concentration of about 10 wt %. In some embodiments, the nanoparticle or composition comprises at least one lipid in a concentration of about 13 wt %. In some embodiments, the nanoparticle or composition comprises at least one lipid in a concentration of about 15 wt %. In some embodiments, the nanoparticle or composition comprises at least one lipid in a concentration of about 20 wt %. In some embodiments, the nanoparticle or composition comprises at least one lipid in a concentration of about 25 wt %. In some embodiments, the nanoparticle or composition comprises at least one lipid in a concentration of about 30 wt %. In some embodiments, the nanoparticle or composition comprises at least one lipid in a concentration of about 35 wt %. In some embodiments, the nanoparticle or composition comprises at least one lipid in a concentration of about 37 wt %. In some embodiments, the nanoparticle or composition comprises at least one lipid in a concentration of about 40 wt %. In some embodiments, the nanoparticle or composition comprises at least one lipid in a concentration of about 45 wt %. In some embodiments, the nanoparticle or composition comprises at least one lipid in a concentration of about 50 wt %. In some embodiments, the nanoparticle or composition comprises at least one lipid in a concentration of about 60 wt %. In some embodiments, the nanoparticle or composition comprises at least one lipid in a concentration of about 70 wt %. In some embodiments, the nanoparticle or composition comprises at least one lipid in a concentration of about 80 wt %. In some embodiments, the nanoparticle or composition comprises at least one lipid in a concentration of about 90 wt %. In some embodiments, the nanoparticle or composition comprises at least one lipid in a concentration of about 95 wt %. In some embodiments, the nanoparticle or composition comprises at least one lipid in a concentration of about 95.5 wt %. In some embodiments, the nanoparticle or composition comprises at least one lipid in a concentration of about 99 wt %. In some embodiments, the nanoparticle or composition comprises at least one lipid in a concentration of about 99.9 wt %. In some embodiments, the nanoparticle or composition comprises at least one lipid in a concentration of about 100 wt %.

### Nanoparticle Helper Compound

In various embodiments, the nanoparticle further comprises at least one helper compound. In some embodiments, the helper compound is a helper lipid, helper polymer, or any combination thereof. In some embodiments, the helper lipid is phospholipid, cholesterol lipid, polymer, cationic lipid, neutral lipid, charged lipid, steroid, steroid analogue, polymer conjugated lipid, stabilizing lipid, or any combination thereof.

In some embodiments, the phospholipid is dioleoyl-phosphatidylethanolamine (DOPE) or a derivative thereof, distearoylphosphatidylcholine (DSPC) or a derivative thereof, distearoyl-phosphatidylethanolamine (DSPE) or a derivative thereof, stearoyloleoylphosphatidylcholine (SOPC) or a derivative thereof, 1-stearioyl-2-oleoyl-phosphatidyethanol amine (SOPE) or a derivative thereof, N-(2,3-dioleoyloxy) propyl)-N,N,N-trimethylammonium chloride (DOTAP) or a derivative thereof, or any combination thereof.

In some embodiments, the cholesterol lipid is cholesterol or a derivative thereof, such as a substituted cholesterol molecule. In some embodiments, the nanoparticle comprises a mixture of cholesterol and a substituted cholesterol molecule.

In some embodiments, the polymer is polyethylene glycol (PEG) or a derivative thereof.

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3-dioleoyloxy) propyl)-N,N,N-trimethylammonium chloride (DOTAP); 3-(N-(N′,N′-dimethylaminoethane)-carbamoyl) cholesterol (DC-Chol), N-(1-(2,3-dioleoyloxy) propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy) propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the cationic lipid is an amino lipid. Representative amino lipids include, but are not limited to, 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA·Cl), 1,2-dilinoleoyl-3-trimethylaminopropane chloride salt (DLin-TAP·Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In some embodiments, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

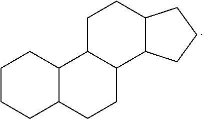 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the stabilizing lipid is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In some embodiments, the polyethylene glycol-lipid is N-[(methoxy poly(ethylene glycol) 2000) carbamyl]-1,2-dimyristyloxlpropyl-3-amine (PEG-c-DMA). In some embodiments, the polyethylene glycol-lipid is PEG-c-DOMG). In other embodiments, the nanoparticles comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy) propyl-1-O-(w-methoxy (polyethoxy)ethyl) butanedioate (PEG-S-DMG), a pegylated ceramide (PEG-cer), or a PEG dialkoxypropylcarbamate such as w-methoxy (polyethoxy)ethyl-N-(2,3-di(tetradecanoxy) propyl) carbamate or 2,3-di(tetradecanoxy) propyl-N-(w-methoxy (polyethoxy)ethyl) carbamate.

In various embodiments, the nanoparticle comprises one or more helper compounds in a concentration range of about 0 mol % to about 100 mol %. In some embodiments, the nanoparticle comprises one or more helper compounds in a concentration range of about 0.01 mol % to about 99.99 mol %. In some embodiments, the nanoparticle comprises one or more helper compounds in a concentration range of about 0.1 mol % to about 99.9 mol %. In some embodiments, the nanoparticle comprises one or more helper compounds in a concentration range of about 0.1 mol % to about 90 mol %. In some embodiments, the nanoparticle comprises one or more helper compounds in a concentration range of about 0.1 mol % to about 70 mol %. In some embodiments, the nanoparticle comprises one or more helper compounds in a concentration range of about 5 mol % to about 95 mol %. In some embodiments, the nanoparticle comprises one or more helper compounds in a concentration range of about 0.5 mol % to about 50 mol %. In some embodiments, the nanoparticle comprises one or more helper compounds in a concentration range of about 0.5 mol % to about 47 mol %. In some embodiments, the nanoparticle comprises one or more helper compounds in a concentration range of about 2.5 mol % to about 47 mol %.

For example, in some embodiments, the nanoparticle comprises one or more helper compounds in a concentration of about 0.01 mol %. In some embodiments, the nanoparticle comprises one or more helper compounds in a concentration of about 0.1 mol %. In some embodiments, the nanoparticle comprises one or more helper compounds in a concentration of about 0.5 mol %. In some embodiments, the nanoparticle comprises one or more helper compound in a concentration of about 1 mol %. In some embodiments, the nanoparticle comprises one or more helper compounds in a concentration of about 1.5 mol %. In some embodiments, the nanoparticle comprises one or more helper compounds in a concentration of about 2 mol %. In some embodiments, the nanoparticle comprises one or more helper compounds in a concentration of about 2.5 mol %. In some embodiments, the nanoparticle comprises one or more helper compounds in a concentration of about 5 mol %. In some embodiments, the nanoparticle comprises one or more helper compound in a concentration of about 10 mol %. In some embodiments, the nanoparticle comprises one or more helper compounds in a concentration of about 12 mol %. In some embodiments, the nanoparticle comprises one or more helper compound in a concentration of about 15 mol %. In some embodiments, the nanoparticle comprises one or more helper compounds in a concentration of about 16 mol %. In some embodiments, the nanoparticle comprises one or more helper compounds in a concentration of about 20 mol %. In some embodiments, the nanoparticle comprises one or more helper compounds in a concentration of about 25 mol %. In some embodiments, the nanoparticle comprises one or more helper compounds in a concentration of about 30 mol %. In some embodiments, the nanoparticle comprises one or more helper compounds in a concentration of about 35 mol %. In some embodiments, the nanoparticle comprises one or more helper compounds in a concentration of about 37 mol %. In some embodiments, the nanoparticle comprises one or more helper compounds in a concentration of about 40 mol %. In some embodiments, the nanoparticle comprises one or more helper compounds in a concentration of about 45 mol %. In some embodiments, the nanoparticle comprises one or more helper compounds in a concentration of about 46.5 mol %. In some embodiments, the nanoparticle comprises one or more helper compounds in a concentration of about 47 mol %. In some embodiments, the nanoparticle comprises one or more helper compounds in a concentration of about 50 mol %. In some embodiments, the nanoparticle comprises one or more helper compounds in a concentration of about 60 mol %. In some embodiments, the nanoparticle comprises one or more helper compounds in a concentration of about 63 mol %. In some embodiments, the nanoparticle comprises one or more helper compounds in a concentration of about 70 mol %. In some embodiments, the nanoparticle comprises one or more helper compounds in a concentration of about 80 mol %. In some embodiments, the nanoparticle comprises one or more helper compounds in a concentration of about 90 mol %. In some embodiments, the nanoparticle comprises one or more helper compounds in a concentration of about 95 mol %. In some embodiments, the nanoparticle comprises one or more helper compounds in a concentration of about 95.5 mol %. In some embodiments, the nanoparticle comprises one or more helper compounds in a concentration of about 99 mol %. In some embodiments, the nanoparticle comprises one or more helper compounds in a concentration of about 100 mol %.

In various aspects, the nanoparticle comprises one or more stabilizers. In some embodiments, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(imides), poly(benzimidazoles), collodion, regenerated proteins, semi-solid cross-linked poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines and carboxylic acids or combinations thereof.

In some embodiments, the nanoparticle comprises one or more molecules selected from the group consisting of phospholipids, albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(3-hydroxyvalerate), poly(D,L-lactide-co-glycolide), poly(l-lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, and any combination thereof.

In some embodiments, the nanoparticle comprises one or more molecules selected from the group consisting of gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, and any combination thereof.

In some embodiments, the nanoparticle is a polymersome. Any polymersome known in the art may be utilized. Thus, In some embodiments, the nanoparticle comprises a homopolymer. In some embodiments, the nanoparticle comprises a block copolymer that is a triblock, tetrablock, pentablock, or at least six block copolymer. In some embodiments, the nanoparticle comprises poly(ethylene oxide) (PEO) block copolymer, poly(ethylethylene) (PEE), poly(butadiene) (PB or PBD), poly(styrene) (PS), poly(isoprene) (PI), PEI, poly(lactide-co-glycolic acid) (PLGA), biodegradable PLGA, polyethylene glycol (PEG), poly(lactide-co-glycolic acid)-polyethylene glycol (PLGA-PEG), poly(lactide-co-glycolic acid)-block-polyethylene glycol (PLGA-b-PEG), biodegradable PLGA-PEG, biodegradable PLGA-b-PEG, polyanhydride, polyanhydride-block-PEG copolymers, zwitterionic poly(carbobetaine), zwitterionic poly(sulfobetaine)-containing, zwitterionic poly(carbobetaine) and zwitterionic poly(sulfobetaine)-containing copolymers, poly(acrylic acid-co-distearin acrylate), poly(trimethylene carbonate)-block-poly(L-glutamic acid), poly(ethylene glycol-block-L-aspartic acid), poly(2-hydroxyethyl-co-octadecyl aspartamide), poly(ethylene glycol-co-trimethylene carbonate-co-caprolactone, polypropylene oxide block copolymers, polyethylene oxide-block-polypropylene oxide copolymers, or any combination thereof.

In some embodiments, the nanoparticle comprises poly(ε-caprolactone) (PCL) diblock co-polymer. In some embodiments, the nanoparticle comprises poly(ethylene oxide)-block-poly(ε-caprolactone) (PEO-b-PCL) based diblock copolymers. In some embodiments, the nanoparticle is derived from the coupling of poly(lactic acid), poly(glycolide), poly(lactic-coglycolic acid) and/or or poly(3-hydroxybutyrate) with PEO. In some embodiments, the nanoparticle comprises PLGA. In some embodiments, the nanoparticle comprises PEG. In some embodiments, the nanoparticle comprises poly(lactide-co-glycolic acid)-polyethylene glycol (PLGA-PEG). For example, in some embodiments, a PLGA-PEG polymersome encapsulates the ICGJ and optionally PEI.

In some embodiments, the nanoparticle further comprises a cationic polymer. The cationic polymer may be a straight chain polymer (i.e., linear polymer) or a branched chain polymer (i.e., branched polymer), including hyperbranched polymers. In some embodiments the cationic polymer is a branched cationic polymer. In some embodiments, the cationic polymer is cross-linked. In some embodiments, the cationic polymer is a polyamine. In some embodiments, the cationic polymer has molecular weight of 5 kDa-3000 kDa. For example, In some embodiments, the cationic polymer has a molecular weight of 5 kDa-2000 kDa, 5 kDa-1500 kDa, 5 kDa-1000 kDa, 5 kDa-800 kDa, 5 kDa-500 kDa, 5 kDa-300 kDa or 5 kDa-200 kDa or 800 kDa-3000 kDa.

In some embodiments, the cationic polymer is a polyalkyleneimine (e.g., polyethyleneimine), polyallylamine, polyamidoamine, or poly(amino-co-ester). In some embodiments, the cationic polymer is polyethyleneimine (PEI), chitosan, poly(2-N,N-dimethylaminoethylmethacrylate), or poly-L-lysine. In some embodiments, the cationic polymer stabilizes the nanocapsule.

In some embodiments, the nanoparticles comprise a biodegradable polymer comprises PLGA, poly(D,L-lactide-co-glycolide), poly(D,L-lactide), poly(D,L-lactide-co-lactide), poly(L-lactide), poly(glycolide), poly(L-lactide-co-glycolide), poly(caprolactone), poly(glycolide-co-trimethylene carbonate), poly(3-hydroxybutyrate), poly(3-hydroxybutyrate-co-3-hydroxyvalerate), poly(4-hydroxybutyrate), poly(ester amide), poly(ester-sulfoester amide), poly(orthoester) or poly(anhydride), and a combination thereof.

In various embodiments, the nanoparticle of the present invention is substantially non-toxic.

In some embodiments, the nanoparticle is a biodegradable nanoparticle. In some embodiments, the nanoparticle is biodegradable nanocapsule. In some embodiments, the nanoparticle is a biodegradable polymer vesicle. In some embodiments, the nanoparticle is a biodegradable liposome.

### Surfactants

In various embodiments, nanoparticles of the invention comprise a surfactant. For example, a nanoparticle of the invention may comprise a targeting agent of the invention, a therapeutic agent, at least one lipid, at least one surfactant, and at least one vitamin E. In some embodiments, the surfactant is any surfactant that is known to those of skill in the art, any emulsifier that is known to those of skill in the art, or any combination thereof. Examples of such surfactants include, but are not limited to, a poloxamer such as a poloxamer of any grade such as poloxamer 188, polyethylene glycol (PEG), functionalized PEG, such as polyethylene glycol 15-hydroxystearate (Kolliphor® HS15), POLYOXYL (40) STEARATE, poly(ethylene glycol)-100-stearate (Myrj59), poly(ethylene glycol)-40-stearate (Myrj 52), Tween® 80 (polysorbate 80), mustard, lecithin, such as soy lecithin and egg lecithin, monoglycerides, diglycerides, polysorbates, carrageenan, guar gum, canola oil, polysorbates (Tween™), sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol (Triton X100™), N, N-dimethyldodecylamine-N-oxide, hexadecyltrimethylammonium bromide (HTAB), polyoxyl 10 lauryl ether, Brij 721™ (poly(ethylene glycol) 21-stearate), bile salts, such as sodium deoxycholate and sodium cholate, polyoxyl castor oil (Cremophor™), nonylphenol ethoxylate (Tergitol™), cyclodextrins, methylbenzethonium chloride (Hyamine™), or a combination thereof. Examples of such emulsifier include, but are not limited to emulsifiers listed in “Emulsifiers: Types and Uses, R Miller, Kansas State University, Manhattan, KS, USA, 2016 Elsevier Ltd. All rights reserved,” incorporated herein by reference in its entirety.

In various embodiments, the nanoparticle comprises at least one surfactant in a concentration range of about 0.1 wt % to about 100 wt %. In some embodiments, the nanoparticle comprises at least one surfactant in a concentration range of about 1 wt % to about 100 wt %. In some embodiments, the nanoparticle comprises at least one surfactant in a concentration range of about 1 wt % to about 70 wt %. In some embodiments, the nanoparticle comprises at least one surfactant in a concentration range of about 10 wt % to about 80 wt %. In some embodiments, the nanoparticle comprises at least one surfactant in a concentration range of about 10 wt % to about 75 wt %. In some embodiments, the nanoparticle comprises at least one surfactant in a concentration range of about 10 wt % to about 70 wt %. In some embodiments, the nanoparticle comprises at least one surfactant in a concentration range of about 10 wt % to about 50 wt %. In some embodiments, the nanoparticle comprises at least one surfactant in a concentration range of about 15 wt % to about 45 wt %. In some embodiments, the nanoparticle comprises at least one surfactant in a concentration range of about 35 wt % to about 40 wt %.

For example, in some embodiments, the nanoparticle comprises at least one surfactant in a concentration of about 1 wt %. In some embodiments, the nanoparticle comprises at least one surfactant in a concentration of about 2 wt %. In some embodiments, the nanoparticle comprises at least one surfactant in a concentration of about 5 wt %. In some embodiments, the nanoparticle comprises at least one surfactant in a concentration of about 5.5 wt %. In some embodiments, the nanoparticle comprises at least one surfactant in a concentration of about 10 wt %. In some embodiments, the nanoparticle comprises at least one surfactant in a concentration of about 13 wt %. In some embodiments, the nanoparticle comprises at least one surfactant in a concentration of about 15 wt %. In some embodiments, the nanoparticle comprises at least one surfactant in a concentration of about 20 wt %. In some embodiments, the nanoparticle comprises at least one surfactant in a concentration of about 25 wt %. In some embodiments, the nanoparticle comprises at least one surfactant in a concentration of about 30 wt %. In some embodiments, the nanoparticle comprises at least one surfactant in a concentration of about 35 wt %. In some embodiments, the nanoparticle comprises at least one surfactant in a concentration of about 37 wt %. In some embodiments, the nanoparticle comprises at least one surfactant in a concentration of about 40 wt %. In some embodiments, the nanoparticle comprises at least one surfactant in a concentration of about 45 wt %. In some embodiments, the nanoparticle comprises at least one surfactant in a concentration of about 50 wt %. In some embodiments, the nanoparticle comprises at least one surfactant in a concentration of about 60 wt %. In some embodiments, the nanoparticle comprises at least one surfactant in a concentration of about 70 wt %. In some embodiments, the nanoparticle comprises at least one surfactant in a concentration of about 80 wt %. In some embodiments, the nanoparticle comprises at least one surfactant in a concentration of about 90 wt %. In some embodiments, the nanoparticle comprises at least one surfactant in a concentration of about 95 wt %. In some embodiments, the nanoparticle comprises at least one surfactant in a concentration of about 95.5 wt %. In some embodiments, the nanoparticle comprises at least one surfactant in a concentration of about 99 wt %. In some embodiments, the nanoparticle comprises at least one surfactant in a concentration of about 99.9 wt %.

### Vitamin Es

In some embodiments, a nanoparticle of the present invention comprises at least one vitamin E. In some embodiments, the vitamin E of a nanoparticle is selected from α-tocopherol, α-tocopherol acetate (αTA), α-tocopherol nicotinate, D-α-tocopheryl polyethylene glycol 1000 succinate, β-tocopherol, β-tocopherol acetate, β-tocopherol nicotinate, γ-tocopherol, γ-tocopherol acetate, γ-tocopherol nicotinate, δ-tocopherol, δ-tocopherol acetate, δ-tocopherol nicotinate, α-tocotrienol, α-tocotrienol acetate, α-tocotrienol nicotinate, β-tocotrienol, β-tocotrienol acetate, β-tocotrienol nicotinate, γ-tocotrienol, γ-tocotrienol acetate, γ-tocotrienol nicotinate, 8-tocotrienol, δ-tocotrienol acetate, δ-tocotrienol nicotinate, and any combination thereof.

In various embodiments, the nanoparticle comprises at least one vitamin E in a concentration range of about 0.1 wt % to about 100 wt %. In some embodiments, the nanoparticle comprises at least one vitamins E in a concentration range of about 1 wt % to about 100 wt %. In some embodiments, the nanoparticle comprises at least one vitamins E in a concentration range of about 1 wt % to about 70 wt %. In some embodiments, the nanoparticle comprises at least one vitamins E in a concentration range of about 5 wt % to about 75 wt %. In some embodiments, the nanoparticle comprises at least one vitamins E in a concentration range of about 10 wt % to about 80 wt %. In some embodiments, the nanoparticle comprises at least one vitamins E in a concentration range of about 10 wt % to about 70 wt %. In some embodiments, the nanoparticle comprises at least one vitamins E in a concentration range of about 10 wt % to about 50 wt %. In some embodiments, the nanoparticle comprises at least one vitamins E in a concentration range of about 15 wt % to about 45 wt %. In some embodiments, the nanoparticle comprises at least one vitamins E in a concentration range of about 35 wt % to about 40 wt %.

For example, in some embodiments, the nanoparticle comprises at least one vitamins E in a concentration of about 1 wt %. In some embodiments, the nanoparticle comprises at least one vitamins E in a concentration of about 2 wt %. In some embodiments, the nanoparticle comprises at least one vitamins E in a concentration of about 5 wt %. In some embodiments, the nanoparticle comprises at least one vitamins E in a concentration of about 5.5 wt %. In some embodiments, the nanoparticle comprises at least one vitamins E in a concentration of about 10 wt %. In some embodiments, the nanoparticle comprises at least one vitamins E in a concentration of about 13 wt %. In some embodiments, the nanoparticle comprises at least one vitamins E in a concentration of about 15 wt %. In some embodiments, the nanoparticle comprises at least one vitamins E in a concentration of about 20 wt %. In some embodiments, the nanoparticle comprises at least one vitamins E in a concentration of about 25 wt %. In some embodiments, the nanoparticle comprises at least one vitamins E in a concentration of about 30 wt %. In some embodiments, the nanoparticle comprises at least one vitamins E in a concentration of about 35 wt %. In some embodiments, the nanoparticle comprises at least one vitamins E in a concentration of about 37 wt %. In some embodiments, the nanoparticle comprises at least one vitamins E in a concentration of about 40 wt %. In some embodiments, the nanoparticle comprises at least one vitamins E in a concentration of about 45 wt %. In some embodiments, the nanoparticle comprises at least one vitamins E in a concentration of about 50 wt %. In some embodiments, the nanoparticle comprises at least one vitamins E in a concentration of about 60 wt %. In some embodiments, the nanoparticle comprises at least one vitamins E in a concentration of about 70 wt %. In some embodiments, the nanoparticle comprises at least one vitamins E in a concentration of about 80 wt %. In some embodiments, the nanoparticle comprises at least one vitamins E in a concentration of about 90 wt %. In some embodiments, the nanoparticle comprises at least one vitamins E in a concentration of about 95 wt %. In some embodiments, the nanoparticle comprises at least one vitamins E in a concentration of about 95.5 wt %. In some embodiments, the nanoparticle comprises at least one vitamins E in a concentration of about 99 wt %. In some embodiments, the nanoparticle comprises at least one vitamins E in a concentration of about 99.9 wt %.

### Conjugates

The present invention also relates, in part, to a conjugate comprising at least one targeting peptide that selectively targets an adipose tissue or ASC and a covalent and/or non-covalent linkage to at least one agent. In some embodiments, the targeting peptide comprises a peptide sequence selected from the group consisting of SEQ ID NO:1, SEQ ID NO:2, SEQ ID NO:3, SEQ ID NO:4, SEQ ID NO:5, and SEQ ID NO:6. In some embodiments, the targeting peptide comprises a peptide sequence of SEQ ID NO:3 and one or more nanoparticle linker sequence positioned at the N-Terminal and/or C-Terminal of the peptide sequence of SEQ ID NO:3.

In various embodiments, the conjugates described herein are formulated for stability for in vivo cell targeting. In some embodiments, the conjugate formulated for stability for in vivo delivery to a tissue or cell of interest.

In some embodiment, the targeting peptide is bound directly to the at least one agent. In some embodiments, the targeting peptide is bound directly to the surface of at least one agent. In some embodiments, the targeting peptide is bound to the at least one agent using a linking molecule. In some embodiments, the targeting peptide is bound to the surface of the at least one agent using a linking molecule.

Linking molecules useful in the conjugates of the present invention may be any molecule capable of binding to both the ligand used in the conjugates of the present invention and the at least one agent used in the conjugates of the present invention. In certain embodiments, the linking molecule may be a hydrophilic polymer. Examples of linking molecules include, but are not limited to, poly(ethylene glycol) and its derivatives, azide compounds, maleimide compounds, hydrazine compounds, dibenzo-cyclooctyne (DBCO) compounds, dithiol compounds, dithiol compounds with hydrazide and/or carboxylic functionality, or single thiols and/or amines, or their derivatives.

In certain embodiments, the linking molecule and the ligand may be bound by one or more covalent bonds. In certain embodiments, the linking molecule, in addition to linking the targeting peptide and the at least one agent, may impart certain benefits upon the conjugates of the present invention, including, but not limited to, improved hydrophilicity and stability in solution, reduced immunogenic responses upon introduction of the conjugates of the present invention into a subject, increased circulation time of the conjugates of the present invention when introduced into the bloodstream of a subject. The choice of a linking molecule may depend upon, among other things, the ligand chosen and the subject into which the conjugates of the present invention are to be introduced. One of ordinary skill in the art, with the benefit of this invention, will recognize additional suitable linking molecules. Such linking molecules are considered to be within the spirit of the present invention.

In some embodiments, the agent is any agent described herein, such as any drug, lipid, polymer, peptide, polypeptide, and/or polypeptide variant described herein. In various embodiments, the conjugate of the present invention is substantially non-toxic. In some embodiments, the conjugate is a biodegradable conjugate.

### Therapeutic Agents

In some aspects, the invention is not limited to any particular cargo or therapeutic agent for which the nanoparticle and/or the conjugate of the invention is able to carry or transport. Rather, the invention includes any therapeutic agent that can be carried by the nanoparticle and/or the conjugate of the invention. For example, the therapeutic agents that can be carried by the nanoparticle and/or the conjugate of the invention include, but are not limited to, diagnostic agents, imaging agents, detectable agents (e.g., dyes), and any therapeutic agents.

In various embodiments, the nanoparticle or any composition of the invention comprises at least one therapeutic agent. In some embodiments, the therapeutic agent is any agent described herein, such as any drug, lipid, polymer, peptide, polypeptide, and/or polypeptide variant described herein.

In some embodiments, the nanoparticle or any composition of the invention comprises a therapeutic agent for delivery to adipose tissue or ASCs. In some embodiments, the nanoparticle comprises resveratrol, or a derivative, prodrug, or pharmaceutically acceptable salt thereof, trans-resveratrol, or a derivative, prodrug, or pharmaceutically acceptable salt thereof, and/or quercetin, or a derivative, prodrug, or pharmaceutically acceptable salt thereof. In some embodiments, the nanoparticle further comprises at least one lipid, at least one surfactant, and at least one vitamin E.

In various embodiments, compositions of the invention increase the concentration of a cellular CAMP in an adipose tissue of interest (e.g., WAT, beige adipose tissue, BAT, etc.) or in ASCs, induces browning of an adipose tissue of interest (e.g., WAT, beige adipose tissue, etc.) or ASCs, induces beiging of ASCs, activates an adipose tissue of interest (e.g., BAT, etc.), increases the level or activity of a 5′ adenosine monophosphate-activated protein kinase (AMPK), increases the level or activity sirtuin 1 (SIRT1), increases the level or activity peroxisome proliferator-activated receptor gamma coacvtivator 1-alpha (PGC-1a), decreases the level or activity of a fatty acid synthase (FAS), decreases the level or activity of acetyl-CoA carboxylase (ACC), decreases the level or activity of NF-κB, decreases the level or activity of interferon-γ, decreases the level or activity of IL-4, decreases the level or activity of IL-1α, decreases the level or activity of a phosphodiesterase (PDE), or a combination thereof. In some embodiments, compositions of the invention increase mitochondrial biogenesis, increases mitochondrial function and thermogenesis, increases gut integrity, increases gut health, increases insulin sensitivity, increases energy expenditure of at least one cell of interest, improves metabolic health, improves gut microbiota profile, improves glucose homeostasis, improves lipid homeostasis, decreases fat mass and body weight, decreases inflammatory response, decreases liver damage, or a combination thereof. In some embodiments, compositions of the invention reduce body fat in a subject. In some embodiments, compositions of the invention induce weight loss in a subject. In some embodiments, compositions of the invention do not change the level or concentration of lean mass.

In some embodiments, the nanoparticle comprises at least one phytochemical or bioactive compound. Examples of such compounds include, but are not limited to, curcumin, theaflavins, thearubigins, epigallocatechin gallate (EGCG), (+)-catechin, (−)-epicatechin, (−)-epicatechin gallate, (−)-epigallocatechin, (+)-gallocatechin, isorhamnetin, kaempferol, myricetin, apigenin, luteolin, baicalein, chrysin, forskolin, chlorophyll a, chlorophyll b, eriodictyol, hesperetin, naringenin, taxifolin, catechins, luteolin, cyanidin, genistein, daidzein, genistein, glycitein, biochanin A, formononetin, cyanidin, delphinidin, malvidin, pelargonidin, peonidin, petunidin, proanthocyanidins, α-carotene, β-carotene, β-cryptoxanthin, lutein, zeaxanthin, lycopene, and any combination thereof.

In some embodiments, the therapeutic agent comprises resveratrol, or a derivative, prodrug, or pharmaceutically acceptable salt thereof, trans-resveratrol, or a derivative, prodrug, or pharmaceutically acceptable salt thereof, quercetin, or a derivative, prodrug, or pharmaceutically acceptable salt thereof.

In some embodiments, the therapeutic agent is adhered to the surface of the nanoparticle and/or the conjugate of the invention. In some embodiments, the therapeutic agent is integrated into the structure of the nanoparticle and/or the conjugate of the invention. In some embodiments, the nanoparticle and/or the conjugate of the invention encapsulates at least one therapeutic agent. In some embodiments, the nanoparticle and/or the conjugate of the invention comprises at least two therapeutic agents.

In some embodiments, the nanoparticle and/or the conjugate of the invention encapsulates one or more therapeutic agents. In some embodiments, the nanoparticle and/or the conjugate of the invention is bound to one or more therapeutic agents. In some embodiments, the therapeutic agent comprises a hydrophobic therapeutic agent. In some embodiments, the therapeutic agent comprises a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics, browning agents, beiging agents, forskolin, resveratrol, quercetin, and vertigo agents, or any combinations thereof.

In some embodiments, the therapeutic agent comprises one or more non-therapeutic moieties. In some embodiments, the nanoparticle and/or the conjugate of the invention comprises one or more therapeutic moieties, one or more non-therapeutic moieties, or any combination thereof.

In some embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In some embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, In some embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene). Additional examples of specific antiproliferative agents include, but are not limited to levamisole, gallium nitrate, granisetron, sargramostim strontium-89 chloride, filgrastim, pilocarpine, dexrazoxane, and ondansetron.

The agent can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In some embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the therapeutic agent comprises one or more gene components, such as siRNA or therapeutic DNA fragments. In some embodiments, the gene component is encapsulated in the nanoparticle. In some embodiments, the gene component is on the surface of the nanoparticle, for example, attached to or within the coating material.

In various embodiments, the therapeutic agent is a small molecule. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In some embodiments, a small molecule therapeutic agent comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In some embodiments, the therapeutic agent is a prodrug. In some embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

In some embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

In other related aspects, the therapeutic agent is a nucleic acid molecule. In various embodiments, the therapeutic agent is an isolated nucleic acid. Thus, in some embodiments, an isolated nucleic acid, including for example a DNA oligonucleotide and an RNA oligonucleotide can be incorporated in the composition of the invention. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In some embodiments, the isolated nucleic acid molecule encodes a therapeutic peptide. In some embodiments, the therapeutic agent is an siRNA, miRNA, shRNA, or an antisense molecule, which inhibits a targeted nucleic acid including those encoding proteins that are involved in aggravation of the pathological processes.

In some embodiments, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells.

In some embodiments, siRNA is used to decrease the level of a targeted protein in a cell, tissue, or whole subject. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Optimizing siRNAs involves consideration of overall G/C content, C/T content at the termini, Tm and the nucleotide content of the 3′ overhang.

In some aspects, the invention includes a vector comprising an siRNA or an antisense polynucleotide. In some embodiments, the siRNA or antisense polynucleotide is capable of inhibiting the expression of a target polypeptide. The incorporation of a desired polynucleotide into a vector and the choice of vectors are well-known in the art.

In certain embodiments, the expression vectors described herein encode a short hairpin RNA (shRNA) therapeutic agent. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in some aspects, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a Tet-on inducible vector for expression in eukaryote cells.

The vector may be obtained by conventional methods known by persons skilled in the art. In a particular embodiment, the vector is a vector useful for transforming animal cells.

In some embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression. The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin, such as IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like.

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl-methyl-, thio- and other modified forms of adenine, cytidine, guanine, thymine, and uridine.

In some embodiments of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art. Antisense nucleic acids are DNA or RNA molecules that are complementary, as that term is defined elsewhere herein, to at least a portion of a specific mRNA molecule. In the cell, antisense nucleic acids hybridize to the corresponding mRNA, forming a double-stranded molecule thereby inhibiting the translation of genes.

The use of antisense methods to inhibit the translation of genes is known in the art. Such antisense molecules may be provided to the cell via genetic expression using DNA encoding the antisense molecule.

Alternatively, antisense molecules of the invention may be made synthetically and then provided to the cell. In some embodiments, antisense oligomers are between about 10 to about 30, or about 15 nucleotides, since they are easily synthesized and introduced into a target cell. Synthetic antisense molecules contemplated by the invention include oligonucleotide derivatives known in the art which have improved biological activity compared to unmodified oligonucleotides.

In some embodiments of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In some embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In some embodiments, the therapeutic agent comprises a gRNA or a nucleic acid molecule encoding a gRNA. In some embodiments, the therapeutic agent comprises a Cas peptide or a nucleic acid molecule encoding a Cas peptide.

In some embodiments, the agent comprises a miRNA or a mimic of a miRNA. In some embodiments, the agent comprises a nucleic acid molecule that encodes a miRNA or mimic of a miRNA.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the therapeutic agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

miRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In some embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells.

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In some embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of an miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In some embodiments, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, at least 70%, at least 85%, or at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. In certain aspects, the identity between two amino acid sequences is determined by using the BLASTN algorithm.

In some embodiments, the nucleic acid molecule is a mRNA molecule. In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the invention.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

In other related aspects, the therapeutic agent is a polypeptide. In various embodiments, the therapeutic agent is an isolated polypeptide. In other related aspects, the therapeutic agent includes an isolated polypeptide. For example, In some embodiments, the therapeutic agent polypeptide of the invention inhibits or activates a target directly by binding to the target thereby modulating the normal functional activity of the target. In some embodiments, the therapeutic agent polypeptide of the invention modulates the target by competing with endogenous proteins. In some embodiments, the therapeutic agent polypeptide of the invention modulates the activity of the target by acting as a transdominant negative mutant.

The variants of the polypeptide therapeutic agents may be (i) one in which one or more of the amino acid residues are substituted with a conserved or non-conserved amino acid residue and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

As used herein, an amino acid sequence is “substantially homologous” to any of the amino acid sequences described herein when its amino acid sequence has a degree of identity with respect to the amino acid sequence of at least 60%, at least 70%, at least 75%, at least 80%, at least 85%, at least 90%, or at least 95%. In some aspects, the identity between two amino acid sequences is determined by using the BLASTP algorithm.

In some embodiments, the therapeutic agent is a peptide. Thus, in some aspects, a peptide therapeutic agent can be incorporated into the nanoparticle and/or the conjugate of the invention. Thus, in some embodiments, the therapeutic agent is a peptide. The peptide therapeutic agent of the present invention may be made using chemical methods. For example, peptides can be synthesized by solid phase techniques, cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The therapeutic agent peptide may alternatively be made by recombinant means or by cleavage from a longer polypeptide. The composition of a therapeutic agent peptide may be confirmed by amino acid analysis or sequencing.

As known in the art the “similarity” between two peptides is determined by comparing the amino acid sequence and its conserved amino acid substitutes of one peptide to a sequence of a second peptide. Variants are defined to include peptide sequences different from the original sequence, for example different from the original sequence in less than 40% of residues per segment of interest, different from the original sequence in less than 25% of residues per segment of interest, different by less than 10% of residues per segment of interest, or different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. In some aspects, the identity between two amino acid sequences is determined by using the BLASTP algorithm.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts to a standard translation reaction.

The therapeutic agent peptides of the invention may include unnatural amino acids formed by post-translational modification or by introducing unnatural amino acids during translation.

In some embodiments, the therapeutic agent is an antibody. Thus, in various embodiments, the composition of the invention comprises an antibody, or antibody fragment. In certain embodiments, the antibody targeting domain specifically binds to a target of interest. In some embodiments, the target of interest is a target at or near the BBB. In some embodiments, the target of interest is a target at or near the BRB. Such antibodies include polyclonal antibodies, monoclonal antibodies, Fab and single chain Fv (scFv) fragments thereof, bispecific antibodies, heteroconjugates, human and humanized antibodies.

The therapeutic agent antibodies may be intact monoclonal or polyclonal antibodies, and immunologically active fragments (e.g., a Fab or (Fab) 2 fragment), an antibody heavy chain, an antibody light chain, humanized antibodies, a genetically engineered single chain Fv molecule, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmacokinetic activity may be generated in a bacterial expression system. Single chain Fv fragments show low immunogenicity.

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology. Further, the antibody of the invention may be “humanized” using the technology described in, and other methods of humanizing antibodies well-known in the art or to be developed.

In some embodiments, a non-human antibody is humanized, where specific sequences or regions of the antibody are modified to increase similarity to an antibody naturally produced in a human or fragment thereof. A humanized antibody can be produced using a variety of techniques known in the art, veneering or resurfacing, chain shuffling. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions.

In some embodiments, the antibody fragment provided herein is a single chain variable fragment (scFv). In various embodiments, the antibodies of the invention may exist in a variety of other forms including, for example, Fv, Fab, and (Fab′) 2, as well as bi-functional (i.e. bi-specific) hybrid antibodies. In some embodiments, the antibodies and fragments thereof of the invention bind a cell bearing antigen, TCR, and/or BCR with wild-type or enhanced affinity. In some embodiments, the antibodies and fragments thereof of the invention bind a T cell bearing TCR with wild-type or enhanced affinity. In some embodiments, the antibodies and fragments thereof of the invention bind a B cell bearing BCR with wild-type or enhanced affinity. In various embodiments, a human scFv may also be derived from a yeast display library.

ScFvs can be prepared according to method known in the art. ScFv molecules can be produced by linking VH and VL regions together using flexible polypeptide linkers. The scFv molecules comprise flexible polypeptide linker (e.g., a Ser-Gly linker) with an optimized length and/or amino acid composition. The flexible polypeptide linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site.

The scFv can comprise a polypeptide linker sequence of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The flexible polypeptide linker sequence may comprise any naturally occurring amino acid. In some embodiments, the flexible polypeptide linker sequence comprises amino acids glycine and serine. In another embodiment, the flexible polypeptide linker sequence comprises sets of glycine and serine repeats such as (Gly4Ser) n, where n is a positive integer equal to or greater than 1. In some embodiments, the flexible polypeptide linkers include, but are not limited to, (Gly4Ser) 4 or (Gly4Ser) 3. Variation in the flexible polypeptide linker length may retain or enhance activity, giving rise to superior efficacy in activity studies.

The invention encompasses monoclonal, synthetic antibodies, and the like. One skilled in the art would understand, based upon the disclosure provided herein, that the crucial feature of the antibody of the invention is that the antibody binds specifically with an antigen of interest. That is, the antibody of the invention recognizes an antigen of interest or a fragment thereof (e.g., an immunogenic portion or antigenic determinant thereof).

In some embodiments, the therapeutic agent is an adjuvant. Thus, in various embodiments, the composition comprises an adjuvant. In some embodiments, the composition comprises a nucleic acid molecule encoding an adjuvant.

Exemplary adjuvants include, but are not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFKB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

In some embodiments, the therapeutic agent is a nucleoside-modified RNA. Thus, In some aspects, the composition comprises a nucleoside-modified RNA. Thus, In some embodiments, the therapeutic agent is a nucleoside-modified RNA. In some embodiments, the composition comprises a nucleoside-modified mRNA. Nucleoside-modified mRNA have particular advantages over non-modified mRNA, including for example, increased stability, low or absent innate immunogenicity, and enhanced translation.

In certain embodiments, nucleoside-modified mRNA does not activate any pathophysiologic pathways, translates very efficiently and almost immediately following delivery, and serve as templates for continuous protein production in vivo lasting for several days. The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects.

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable.

It has been demonstrated that the presence of modified nucleosides, including pseudouridines in RNA suppress their innate immunogenicity. Further, protein-encoding, in vitro-transcribed RNA containing pseudouridine can be translated more efficiently than RNA containing no or other modified nucleosides. Subsequently, it is shown that the presence of pseudouridine improves the stability of RNA and abates both activation of PKR and inhibition of translation. A preparative HPLC purification procedure has been established that was critical to obtain pseudouridine-containing RNA that has superior translational potential and no innate immunogenicity. Administering HPLC-purified, pseudourine-containing RNA coding for erythropoietin into mice and macaques resulted in a significant increase of serum EPO levels, thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In some embodiments, the nucleoside-modified RNA of the invention is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In some embodiments, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the modified nucleoside of the present invention is m⁵ C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²io⁶A (2-methylthio-N⁶-isopentenyladenosine); io⁶A (N⁶-(cis-hydroxyisopentenyl) adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac4C (N⁴-acetylcytidine); f⁵C (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr (p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl) uridine); ho5U (5-hydroxyuridine); mo5U (5-methoxyuridine); cmo⁵U (uridine 5-oxyacetic acid); mcmo5U (uridine 5-oxyacetic acid methyl ester); chm5U (5-(carboxyhydroxymethyl) uridine)); mchm5U (5-(carboxyhydroxymethyl) uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C (5-hydroxymethylcytidine); m³U (3-methyluridine); cm5U (5-carboxymethyluridine); m⁶Am (N⁶,2′-O-dimethyladenosine); m⁶₂Am (N⁶,N⁶,O-2′-trimethyladenosine); m^{2,7}G (N^{2,7}-dimethylguanosine); m^{2,2,7}G (N²,N^{2,7}-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a three-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified RNA of the present invention exhibits significantly less innate immunogenicity than an unmodified in vitro-synthesized RNA molecule with the same sequence. In another embodiment, the modified RNA molecule exhibits an innate immune response that is 2-fold less than its unmodified counterpart. In another embodiment, innate immunogenicity is reduced by a three-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the recombinant protein. In another embodiment, the decrease is such that the nucleoside-modified RNA can be repeatedly administered without eliciting an innate immune response sufficient to eliminate detectable production of the recombinant protein.

In some embodiments, the therapeutic agent is a detectable agent. In some embodiments, the detectable agent is a dye. In some embodiments, the therapeutic agent is a diagnostic agent. In some embodiments, the diagnostic agent is a dye.

In some embodiments, the nanoparticle and/or the conjugate of the invention is bound to a dye. In some embodiments, the nanoparticle and/or the conjugate of the invention encapsulates a dye. In some embodiments, the dye is a polymethine dye. In some embodiments, the polymethine dye can be a cyanine dye, hemicyanine dye, streptocyanine dye, mercocyanine dye, oxonol dye, styryl dye, diarylmethine dye, triarylmethine dye, rylenes, squaraines, and perylene bismides and aza-analogs thereof. In some embodiments, the dye is a cyanine dye. Exemplary cyanine dyes include, but are not limited to, naphthalocyanine dyes, sulfonated indocyanines, indocyanine green, Cy3, Cy3.5, Cy5.5, and Cy7. In some embodiments, the dye is a merocyanine. Exemplary merocyanine dyes include, but are not limited to, pseudoisocyanine chloride and merocyanine I. In some embodiments, the dye is a squaraine. Exemplary squaraine dyes include, but are not limited to, squarylium dye III. In some embodiments, the dye is a rylene. Exemplary rylene dyes include, but are not limited to, bismide. In some embodiments, the dye is indocyanine green (ICG). In some embodiments, the dye is Congo Red. In some embodiments, the dye is IR783. In some embodiments, the dye is Brilliant Blue G. In some embodiments, the dye is rhodamine 6G.

In some embodiments, the dye has an absorbance in the near infrared (NIR) range between about 650 and 1400 nm. In some embodiments, the dye has an absorbance in the NIR range between about 680 and 1100 nm. In some embodiments, the dye has an absorbance in the NIR range between about 700 and 950 nm. In some embodiments, the dye has an absorbance in the NIR range between about 715 and 950 nm. In some embodiments, the dye has an absorbance in the NIR range between about 790 and 895 nm.

In some embodiments, the nanoparticle and/or the conjugate of the invention comprises a dye that has at least one absorption peak from about 650 to 1400 nm wavelength. In some embodiments, the nanoparticle and/or the conjugate of the invention encapsulating a dye has at least one absorption peak from about 650 to 1400 nm wavelength. In some embodiments, the absorption peak is from about 680 to 1100 nm wavelength. In some embodiments, the absorption peak is from about 715 to 950 nm wavelength. In some embodiments, the absorption peak from about 790 to 895 nm wavelength.

In some embodiments, the nanoparticle and/or the conjugate of the invention comprises at least one imaging agent. Imaging agents are materials that allow the microcarrier to be visualized after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting may be used in nanoparticles and/or the conjugate of the invention.

Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Imaging agents must reach their targets to be effective; thus, in some embodiments, an efficient uptake by cells is desirable. A rapid uptake may also be desirable to avoid the RES.

Further, imaging agents should provide high signal to noise ratios so that they may be detected in small quantities, whether directly, or by effective amplification techniques that increase the signal associated with a particular target. Amplification strategies may include, for example, avidin-biotin binding systems, trapping of converted ligands, probes that change physical behavior after being bound by a target, and taking advantage of relaxation rates. Examples of imaging technologies include magnetic resonance imaging, radionuclide imaging, computed tomography, ultrasound, and optical imaging.

Nanoparticles and/or conjugates as set forth herein may be advantageously used in various imaging technologies or strategies, for example by incorporating imaging agents into the nanoparticles and/or conjugates. Many imaging techniques and strategies are known; such strategies may be adapted to use with nanoparticles and/or conjugates. Suitable imaging agents include, for example, fluorescent molecules, labeled antibodies, labeled avidin: biotin binding agents, colloidal metals (e.g., gold, silver), reporter enzymes (e.g., horseradish peroxidase), superparamagnetic transferrin, second reporter systems (e.g., tyrosinase), and paramagnetic chelates. Advantages of nanoparticles and/or conjugates less than about 100 nm in diameter include for example, the ability of the nanoparticles and/or conjugates to be readily delivered and taken up by cells.

Compared to imaging agents that are merely conjugated to a targeting molecule, nanoparticles and/or conjugates can increase signal-to-noise ratio by delivering larger imaging agent loads per uptake event resulting in higher amplification. Many imaging agents may be loaded into the nanoparticle and/or the conjugate of the invention having a targeting molecule, which passes into cell via a single uptake.

In some embodiments, the imaging agent is a magnetic resonance imaging contrast agent. Examples of magnetic resonance imaging contrast agents include, but are not limited to, 1,4,7,10-tetraazacyclododecane-N,N′,N″N″-tetraacetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (DOTA) and derivatives thereof. In some embodiments, the imaging agent is an X-Ray contrast agent. X-ray contrast agents already known in the art include a number of halogenated derivatives, especially iodinated derivatives, of 5-amino-isophthalic acid.

Clinical imaging is of increasing helpfulness in clinical and research settings. Current uses include laboratory medicine, surgery, radiation therapy, nuclear medicine, and diagnostic radiology. Nanoparticles and/or conjugates may be loaded with agents that enhance these processes, for example by enhancing contrast, or delivering agents to cells that allow for visualization with such techniques.

In some embodiments, the composition of the present invention comprises a combination of therapeutic agents described herein. In certain embodiments, a composition comprising a combination of therapeutic agents described herein has an additive effect, wherein the overall effect of the combination is approximately equal to the sum of the effects of each individual therapeutic agents. In other embodiments, a composition comprising a combination of therapeutic agents described herein has a synergistic effect, wherein the overall effect of the combination is greater than the sum of the effects of each individual therapeutic agent.

A composition comprising a combination of therapeutic agents comprises individual agents in any suitable ratio. For example, In some embodiments, the composition comprises a 1:1 ratio of two individual therapeutic agents. However, the combination is not limited to any particular ratio. Rather any ratio that is shown to be effective is encompassed.

In various embodiments, resveratrol, or the derivative, prodrug, or pharmaceutically acceptable salt thereof, is encapsulated within the nanoparticle, adhered to the surface of the nanoparticle, integrated into the structure of the nanoparticle, bound to the nanoparticle, or any combination thereof. In some embodiments, resveratrol, or the derivative, prodrug, or pharmaceutically acceptable salt thereof, coats the outside surface of the nanoparticle. In some embodiments, resveratrol, or the derivative, prodrug, or pharmaceutically acceptable salt thereof, coats a portion of the outside surface of the nanoparticle.

In various embodiments, the nanoparticle or composition comprises resveratrol, or a derivative, prodrug, or pharmaceutically acceptable salt thereof, in a concentration range of about 0.01 wt % to about 99.99 wt %. In some embodiments, the nanoparticle or composition comprises resveratrol, or a derivative, prodrug, or pharmaceutically acceptable salt thereof, in a concentration range of about 0.1 wt % to about 99.9 wt %. In some embodiments, the nanoparticle or composition comprises resveratrol, or a derivative, prodrug, or pharmaceutically acceptable salt thereof, in a concentration range of about 1 wt % to about 70 wt %. In some embodiments, the nanoparticle or composition comprises resveratrol, or a derivative, prodrug, or pharmaceutically acceptable salt thereof, in a concentration range of about 1 wt % to about 60 wt %. In some embodiments, the nanoparticle or composition comprises resveratrol, or a derivative, prodrug, or pharmaceutically acceptable salt thereof, in a concentration range of about 1 wt % to about 55 wt %. In some embodiments, the nanoparticle or composition comprises resveratrol, or a derivative, prodrug, or pharmaceutically acceptable salt thereof, in a concentration range of about 0.1 wt % to about 50 wt %. In some embodiments, the nanoparticle or composition comprises resveratrol, or a derivative, prodrug, or pharmaceutically acceptable salt thereof, in a concentration range of about 1 wt % to about 50 wt %. In some embodiments, the nanoparticle or composition comprises resveratrol, or a derivative, prodrug, or pharmaceutically acceptable salt thereof, in a concentration range of about 1 wt % to about 10 wt %.

For example, in some embodiments, the nanoparticle or composition comprises resveratrol, or a derivative, prodrug, or pharmaceutically acceptable salt thereof, in a concentration of about 0.01 wt %. In some embodiments, the nanoparticle or composition comprises resveratrol, or a derivative, prodrug, or pharmaceutically acceptable salt thereof, in a concentration of about 0.02 wt %. In some embodiments, the nanoparticle or composition comprises resveratrol, or a derivative, prodrug, or pharmaceutically acceptable salt thereof, in a concentration of about 0.05 wt %. In some embodiments, the nanoparticle or composition comprises resveratrol, or a derivative, prodrug, or pharmaceutically acceptable salt thereof, in a concentration of about 0.15 wt %. In some embodiments, the nanoparticle or composition comprises resveratrol, or a derivative, prodrug, or pharmaceutically acceptable salt thereof, in a concentration of about 1.0 wt %. In some embodiments, the nanoparticle or composition comprises resveratrol, or a derivative, prodrug, or pharmaceutically acceptable salt thereof, in a concentration of about 7 wt %. In some embodiments, the nanoparticle or composition comprises resveratrol, or a derivative, prodrug, or pharmaceutically acceptable salt thereof, in a concentration of about 10 wt %. In some embodiments, the nanoparticle or composition comprises resveratrol, or a derivative, prodrug, or pharmaceutically acceptable salt thereof, in a concentration of about 20 wt %. In some embodiments, the nanoparticle or composition comprises resveratrol, or a derivative, prodrug, or pharmaceutically acceptable salt thereof, in a concentration of about 30 wt %. In some embodiments, the nanoparticle or composition comprises resveratrol, or a derivative, prodrug, or pharmaceutically acceptable salt thereof, in a concentration of about 40 wt %. In some embodiments, the nanoparticle or composition comprises resveratrol, or a derivative, prodrug, or pharmaceutically acceptable salt thereof, in a concentration of about 50 wt %.

In various embodiments, quercetin, or the derivative, prodrug, or pharmaceutically acceptable salt thereof, is encapsulated within the nanoparticle, adhered to the surface of the nanoparticle, integrated into the structure of the nanoparticle, bound to the nanoparticle, or any combination thereof. In some embodiments, quercetin, or the derivative, prodrug, or pharmaceutically acceptable salt thereof, coats the outside surface of the nanoparticle. In some embodiments, quercetin, or the derivative, prodrug, or pharmaceutically acceptable salt thereof, coats a portion of the outside surface of the nanoparticle.

In various embodiments, the nanoparticle or composition comprises quercetin, or a derivative, prodrug, or pharmaceutically acceptable salt thereof, in a concentration range of about 0.01 wt % to about 99.99 wt %. In some embodiments, the nanoparticle or composition comprises quercetin, or a derivative, prodrug, or pharmaceutically acceptable salt thereof, in a concentration range of about 0.1 wt % to about 99.9 wt %. In some embodiments, the nanoparticle or composition comprises quercetin, or a derivative, prodrug, or pharmaceutically acceptable salt thereof, in a concentration range of about 1 wt % to about 70 wt %. In some embodiments, the nanoparticle or composition comprises quercetin, or a derivative, prodrug, or pharmaceutically acceptable salt thereof, in a concentration range of about 1 wt % to about 60 wt %. In some embodiments, the nanoparticle or composition comprises quercetin, or a derivative, prodrug, or pharmaceutically acceptable salt thereof, in a concentration range of about 1 wt % to about 55 wt %. In some embodiments, the nanoparticle or composition comprises quercetin, or a derivative, prodrug, or pharmaceutically acceptable salt thereof, in a concentration range of about 0.1 wt % to about 50 wt %. In some embodiments, the nanoparticle or composition comprises quercetin, or a derivative, prodrug, or pharmaceutically acceptable salt thereof, in a concentration range of about 1 wt % to about 50 wt %. In some embodiments, the nanoparticle or composition comprises quercetin, or a derivative, prodrug, or pharmaceutically acceptable salt thereof, in a concentration range of about 1 wt % to about 10 wt %.

For example, in some embodiments, the nanoparticle or composition comprises quercetin, or a derivative, prodrug, or pharmaceutically acceptable salt thereof, in a concentration of about 0.01 wt %. In some embodiments, the nanoparticle or composition comprises quercetin, or a derivative, prodrug, or pharmaceutically acceptable salt thereof, in a concentration of about 0.02 wt %. In some embodiments, the nanoparticle or composition comprises quercetin, or a derivative, prodrug, or pharmaceutically acceptable salt thereof, in a concentration of about 0.05 wt %. In some embodiments, the nanoparticle or composition comprises quercetin, or a derivative, prodrug, or pharmaceutically acceptable salt thereof, in a concentration of about 0.15 wt %. In some embodiments, the nanoparticle or composition comprises quercetin, or a derivative, prodrug, or pharmaceutically acceptable salt thereof, in a concentration of about 1.0 wt %. In some embodiments, the nanoparticle or composition comprises quercetin, or a derivative, prodrug, or pharmaceutically acceptable salt thereof, in a concentration of about 7 wt %. In some embodiments, the nanoparticle or composition comprises quercetin, or a derivative, prodrug, or pharmaceutically acceptable salt thereof, in a concentration of about 10 wt %. In some embodiments, the nanoparticle or composition comprises quercetin, or a derivative, prodrug, or pharmaceutically acceptable salt thereof, in a concentration of about 20 wt %. In some embodiments, the nanoparticle or composition comprises quercetin, or a derivative, prodrug, or pharmaceutically acceptable salt thereof, in a concentration of about 30 wt %. In some embodiments, the nanoparticle or composition comprises quercetin, or a derivative, prodrug, or pharmaceutically acceptable salt thereof, in a concentration of about 40 wt %. In some embodiments, the nanoparticle or composition comprises quercetin, or a derivative, prodrug, or pharmaceutically acceptable salt thereof, in a concentration of about 50 wt %.

### Coating Agents

In various embodiments, the nanoparticle or composition further comprises at least one coating agent. In some embodiments, the coating agent coats the outside surface of the nanoparticle. In some embodiments, the coating agent coats a portion of the outside surface of the nanoparticle. In some embodiments, the coating agent comprises a starch, stabilizer, plasticizer, lipid, polymer, polysaccharide, protein, zein, soy protein, whey, casein, fatty acid, wax, neutral lipid, resin, cellulose, chitosan, alginate, starch, or any combination thereof.

In some embodiments, the coating agent comprises a biocompatible polymer. Examples of coating agents include, but are not limited to, biocompatible polymer, a biodegradable polymer, a multifunctional linker, starch, modified starch, and starch derivatives, gums, including but not limited to polymers, polypeptides, albumin, amino acids, alcohols (e.g., PVA, ethyl alcohol, etc.), thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, phospholipids, albumin, dextran, gelatin, poly(ethylene glycerol) (PEG), poly(vinylpyrrolidone), hyaluronic acid, heparin, heparin sulfate, sialic acid, poly(N-acetylglucosamine) (Chitin), Chitosan, poly(3-hydroxyvalerate), poly(D,L-lactide-co-glycolide), poly(l-lactide-co-glycolide), poly(3-hydroxybutyrate), poly(4-hydroxybutyrate), poly(3-hydroxybutyrate-co-3-hydroxyvalerate), polyorthoester, polyanhydride, poly(glycolic acid), poly(glycolide), poly(L-lactic acid), poly(L-lactide), poly(D,L-lactic acid), poly(D,L-lactide), poly(L-lactide-co-D,L-lactide), poly(caprolactone), poly(L-lactide-co-caprolactone), poly(D,L-lactide-co-caprolactone), poly(glycolide-co-caprolactone), poly(trimethylene carbonate), polyester amide, poly(glycolic acid-co-trimethylene carbonate), co-poly(ether-esters) (e.g. PEO/PLA), polyphosphazenes, fibrin, fibrin glue, fibrinogen, cellulose, starch, collagen and hyaluronic acid, elastin and hyaluronic acid, polyurethanes, silicones, polyesters, polyolefins, polyisobutylene and ethylene-alphaolefin copolymers, acrylic polymers and copolymers other than polyacrylates, vinyl halide polymers and copolymers, polyvinyl chloride, polyvinyl ethers, polyvinyl methyl ether, polyvinylidene halides, polyvinylidene chloride, poly(vinylidene fluoride), poly(vinylidene fluoride-co-hexafluoropropylene), polyacrylonitrile, polyvinyl ketones, polyvinyl aromatics, polystyrene, polyvinyl esters, polyvinyl acetate, acrylonitrile-styrene copolymers, ABS resins, polyamides, Nylon 66, polycaprolactam, polycarbonates including tyrosine-based polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, carboxymethyl cellulose, fullerenes, lipids, or any combination thereof.

In some embodiments, the coating agent comprises at least one molecules selected from gelatin, albumin, dextrose, dextran, a high molecular weight poly(ethylene glycol) or a high molecular weight poly(vinylpyrrolidone), hyaluronic acid, heparin, heparin sulfate, sialic acid, Chitosan, or any combination thereof.

In some embodiments, the nanoparticle or composition further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle or composition taking into consideration at least the type of therapy to be performed.

### Secondary Targeting Agents

In some embodiments, a nanoparticle of the present invention comprises a second targeting agent. The second targeting agent may target or recognize any particular site of interest in a subject.

In various embodiments, the second targeting agent is an antibody, an antibody fragment, a peptide sequence, aptamer, folate, a ligand, a gene component, or any combination thereof. Examples of second targeting agents include, but are not limited to antibodies, lymphokines, cytokines, receptor proteins such as CD4 and CD8, solubilized receptor proteins such as soluble CD4, hormones, growth factors, peptidomimetics, synthetic ligands, and the like which specifically bind desired target cells, and nucleic acids which bind corresponding nucleic acids through base pair complementarity. Second targeting agents of particular interest include peptidomimetics, peptides, aptamers, folates, ligands, gene components, antibodies (e.g., monoclonal antibodies, polyclonal antibodies, recombinant antibodies, human antibodies, humanized antibodies, single domain antibodies (nanobodies), etc.) and antibody fragments (e.g., the Fab′ fragment).

In certain embodiments, the second targeting agent specifically binds to a tumor-associated antigen (TAA) or tumor specific antigen (TSA). Cellular targets include tissue specific cell surface molecules, for targeting to specific sites of interest (e.g., neural cells, liver cells, bone marrow cells, kidney cells, pancreatic cells, muscle cells, adipose cells, ASCs and the like).

Methods of making and using antibodies are well known in the art. For example, polyclonal antibodies useful in the present invention are generated by immunizing rabbits according to standard immunological techniques well-known in the art. Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies, inter alia, which bind to the specific antigens of interest.

One skilled in the art would appreciate, based upon the disclosure provided herein, that the antibody can specifically bind with any portion of an antigen target, which can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest can be used to produce antibodies that are specific only for that antigen and do not cross-react non-specifically with other proteins.

In some embodiments, the second targeting agent is bound directly to the nanoparticle and/or the conjugate of the invention. In some embodiments, the second targeting agent is bound directly to the surface of the nanoparticle and/or the conjugate.

In some embodiments, the second targeting agent is bound to the nanoparticle and/or the conjugate using a linking molecule. In some embodiments, the targeting domain is bound to the surface of the nanoparticle and/or the conjugate using a linking molecule. The linking molecules useful in the nanoparticles, compositions, and methods of the present disclosure may be any molecule capable of binding to both the coating material used in the nanoparticles, compositions, and methods of the present disclosure and the targeting domains used in the nanoparticles, compositions, and methods of the present disclosure. In certain embodiments, the linking molecule may be a hydrophilic polymer. Examples of linking molecules include, but are not limited to, poly(ethylene glycol) and its derivatives, azide compounds, maleimide compounds, hydrazine compounds, dibenzo-cyclooctyne (DBCO) compounds, dithiol compounds, dithiol compounds with hydrazide and/or carboxylic functionality, or single thiols and/or amines or their derivatives.

The second targeting agent may be incorporated in the nanoparticle by any method or configuration described for targeting agents herein.

In certain embodiments, the second targeting agent may be chosen, among other things, to at least partially increase the uptake of the nanoparticle of the present disclosure into a desired cell and/or tissue type when introduced into a subject. In certain embodiments, the second targeting agent may be a moiety that recognizes a molecule which is present in higher amounts in an abnormal form of a tissue when compared to a normal form of the same tissue (i.e., the molecule is “up-regulated” in the abnormal form of the tissue).

In some embodiments, the suitable second targeting agent may be a peptide sequence, DNA fragment, aptamer, RNA, folate, polymer, etc. One of ordinary skill in the art, with the benefit of this disclosure, will recognize other targeting domains that may be useful in the nanoparticle of the present disclosure. Such targeting domains are considered to be within the spirit of the present disclosure.

### Compositions

In some aspects, the present invention relates to a composition comprising at least one nanoparticle and/or at least one conjugate of the present invention. In some aspects, the present invention relates to a composition comprising at least one nanoparticle comprising a targeting peptide of the invention and/or at least one conjugate comprising a targeting peptide of the invention. Thus, in certain embodiments, the composition targets at least one cell comprising a ΔDCN receptor. For example, in some embodiments, the composition targets at least one ASC.

In some embodiments, the composition comprises at least two different nanoparticles comprising different therapeutic agents or targeting agents, at least two different conjugates comprising different therapeutic agents or targeting agents, or any combination thereof. In some embodiments, the composition comprises multiple nanoparticles, multiple conjugates, or any combination thereof.

### Pharmaceutical Compositions

In some embodiments, a composition of the present invention is a pharmaceutical composition. The formulations of the pharmaceutical compositions described herein may be prepared by any method known or hereafter developed in the art of pharmacology. In general, such preparatory methods include the step of bringing the active ingredient into association with a carrier or at least one other accessory ingredient, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise at least one additional pharmaceutically active agent.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intrafat, transdermal delivery, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise at least one additional ingredient including, but not limited to, suspending, stabilizing, or dispersing agents. In some embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. In some embodiments, such powders comprise particles wherein at least 98% of the particles by weight have a diameter greater than 0.5 nanometers and at least 95% of the particles by number have a diameter less than 7 nanometers. In some embodiments, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some aspects, having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise at least one additional ingredient including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, at least one of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Methods

In some aspects, the present invention provides a method of delivering a targeting peptide, nanoparticle, and/or composition of the invention to a subject in need thereof. In some embodiments, a subject in need has at least one metabolic disease or disorder, at least one cancer, or wrinkle formation.

In some embodiments, the present invention provides a method of delivering a targeting peptide, nanoparticle, and/or composition of the invention to an adipose tissue of interest (e.g., WAT, beige adipose tissue, BAT, etc.) and/or ASCs. In some embodiments, the present invention provides a method for in vivo delivery of a targeting peptide, nanoparticle, and/or composition of the invention to an adipose tissue of interest (e.g., WAT, beige adipose tissue, BAT, etc.) and/or ASCs.

In some embodiments, the present invention provides a method of treating a metabolic disease or disorder in a subject in need thereof, comprising administering to a subject a targeting peptide, nanoparticle, and/or composition of the invention. In some embodiments, the metabolic disease or disorder is selected from obesity, diabetes, heart disease, stroke, cardiovascular disease, hypertension, idiopathic intracranial hypertension, coronary artery disease, atrial fibrillation, atherosclerosis, venous thromboembolism, obstructive sleep apnea, obesity hypoventilation syndrome, metabolic syndrome, insulin resistance, non-alcoholic fatty liver disease, or any combination thereof. In some embodiments, the metabolic disease or disorder is obesity.

In some embodiments, the present invention provides a method of treating cancer in a subject in need thereof comprising administering to the subject a targeting peptide, nanoparticle, and/or composition of the invention. In some embodiments, the cancer is selected from the group consisting of esophageal adenocarcinoma, gastric cancer, colorectal cancer, hepatocellular carcinoma, cholangiocarcinoma, pancreatic cancer, endometrial carcinoma, ovarian cancer, breast cancer, renal cell carcinoma, multiple myeloma, or any combination thereof.

In some embodiments, the present invention provides a method of reducing or preventing wrinkle formation in a subject in need thereof comprising administering to the subject a targeting peptide, nanoparticle, and/or composition of the invention.

In some embodiments, the present invention relates to a method of inducing browning of an adipose tissue, beiging of an adipose tissue, browning of ASCs, and/or beiging of ASCs. In some embodiments, the invention provides a method of inducing browning and/or beiging of adipose tissue and/or ASCs in a subject in need thereof, wherein the method comprises administering to the subject: at least one targeting peptide, nanoparticle, and/or composition of the invention.

In some embodiments, the present invention provides a method of reducing body fat in a subject in need thereof, wherein the method comprises administering to the subject: at least one targeting peptide, nanoparticle, and/or composition of the invention.

In some embodiments, the present invention provides a method of inducing weight loss in a subject in need thereof, wherein the method comprises administering to the subject: at least one targeting peptide, nanoparticle, and/or composition of the invention.

In some embodiments, the present invention provides a method of treating or preventing a metabolic disease or disorder in a subject in need thereof, wherein the method comprises administering to the subject at least one targeting peptide, nanoparticle, and/or composition of the invention.

In some embodiments, the method further comprises a fecal microbiota transplantation. In some embodiments, the fecal microbiota transplantation is a human fecal microbiota transplantation (HFT).

In some embodiments, the present invention provides a method of optimizing or improving the gut microbiota in a subject in need thereof, wherein the method comprises administering to the subject: a) a fecal microbiota transplantation; and b) at least one composition comprising a targeting peptide of the invention.

In various embodiments, the method comprises administering an effective amount of a targeting peptide, nanoparticle, and/or composition of the invention to a subject in need thereof. In various embodiments, the method comprises administering an effective amount of a therapeutic agent to a subject in need thereof comprising the step of administering a targeting peptide, nanoparticle, and/or composition of the invention and a therapeutic agent to a subject in need thereof.

In some embodiments, the nanoparticle and/or composition of the invention comprises the therapeutic agent. In some embodiments, the method comprises subcutaneously injecting an effective amount of a nanoparticle and/or composition of the invention into an adipose tissue of interest (e.g., WAT, beige adipose tissue, BAT, etc.) of a subject. In some embodiments, the method comprises injecting an effective amount of a nanoparticle and/or composition of the invention into an adipose tissue of interest (e.g., WAT, beige adipose tissue, BAT, etc.). In various embodiments, the method comprises administering an effective amount of at least one nanoparticle and/or composition of the invention to a subject in need.

In some embodiments, the method comprises administration of the nanoparticle and/or composition of the invention to a subject. In certain embodiments, the method comprises administering a plurality of doses to the subject. In another embodiment, the method comprises administering a single dose of the nanoparticle and/or composition, where the single dose is effective in delivery of the therapeutic agent to a target tissue or target cell.

Administration of the nanoparticles and/or compositions of the invention in a method of treatment can be achieved in a number of different ways, using methods known in the art. In some embodiments, the method of the invention comprises systemic administration of the subject. Examples of suitable routes of administration include, but are not limited to, enteral or parenteral administration, oral administration, injection, or a combination thereof. In some embodiments, the method comprises intrafat delivery of the nanoparticle and/or composition. In certain embodiments, the method comprises intradermal delivery of the nanoparticle and/or composition. In another embodiment, the method comprises intravenous delivery of the nanoparticle and/or composition. In some embodiments, the method comprises intramuscular delivery of the nanoparticle and/or composition. In some embodiments, the method comprises subcutaneous delivery of the nanoparticle and/or composition. In some embodiments, the method comprises inhalation of the nanoparticle and/or composition. In some embodiments, the method comprises intranasal delivery of the nanoparticle and/or composition. In some embodiments, the method comprises transdermal delivery of the nanoparticle and/or composition.

In some embodiments, administration comprises intravenous, intranasal, intramuscular, subcutaneous, transdermal, or intrafat delivery of a composition comprising at least one targeting peptide of the present invention and a therapeutic agent.

It will be appreciated that the composition of the invention may be administered to a subject either alone, or in conjunction with another agent.

The nanoparticles and/or compositions useful for practicing the invention may be administered to deliver a dose of from 0.1 ng/kg/day and 100 mg/kg/day. In some embodiments, the invention envisions administration of a dose which results in a concentration of the compound of the present invention from 10 nM and 10 μM in a mammal.

Typically, dosages which may be administered in a method of the invention to a mammal, for example a human, range in amount from 0.01 μg to about 1 g per kilogram of body weight of the mammal, while the precise dosage administered will vary depending upon any number of factors, including but not limited to, the type of mammal and type of disease state being treated, the age of the mammal and the route of administration. In some embodiments, the dosage of the compound will vary from about 0.1 μg to about 100 mg per kilogram of body weight of the mammal. In some embodiments, the dosage of the compound will vary from about 0.1 μg to about 50 mg per kilogram of body weight of the mammal. In some embodiments, the dosage of the compound will vary from about 0.1 μg to about 10 mg per kilogram of body weight of the mammal. In some embodiments, the dosage will vary from about 1 μg to about 1 mg per kilogram of body weight of the mammal.

The nanoparticles and/or compositions may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In certain embodiments, administration of a nanoparticle and/or composition of the present invention may be performed by single administration or boosted by multiple administrations.

In some embodiments, the invention includes a method comprising administering a combination of nanoparticles and/or compositions described herein. In certain embodiments, the combination has an additive effect, wherein the overall effect of the administering the combination is approximately equal to the sum of the effects of administering each individual nanoparticle and/or composition. In other embodiments, the combination has a synergistic effect, wherein the overall effect of administering the combination is greater than the sum of the effects of administering each individual nanoparticle and/or composition.

In some embodiments, the nanoparticles and/or compositions of the invention may be administered prophylactically (i.e., to prevent obesity or a metabolic disease or disorder) or therapeutically (i.e., to treat obesity or a metabolic disease or disorder) to subjects suffering from or at risk of (or susceptible to) developing the obesity or a metabolic disease or disorder. Such subjects may be identified using standard clinical methods.

In the context of the present invention, prophylactic administration occurs prior to the manifestation of overt clinical symptoms of a metabolic disease or disorder, such that metabolic disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The nanoparticles and/or compositions of the invention can be useful in combination with therapeutic, anti-cancer, and/or radiotherapeutic agents. Thus, the present disclosure provides a combination of the present nanoparticle with therapeutic, anti-cancer, and/or radiotherapeutic agents for simultaneous, separate, or sequential administration. The composition of the invention and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Adipose Stromal Stem Cell-Targeted Nanoparticles: Targeted Delivery of Resveratrol for Inducing Beige Adipocyte Formation

Adipose stromal cells (ASCs) have the potential to differentiate into many different cell types. For example, ASCs can be induced to differentiate into brown-like/beige adipocytes to treat obesity. There is a critical need for effective, safe, and ASC-targeted anti-obesity therapeutics. Inducing the browning of subcutaneous (subQ) white adipose tissue (WAT) to promote thermogenesis is a promising strategy for combating obesity. However, technical breakthroughs in this arena have been limited, and human studies often yield inconclusive results.

Many browning agents have the potential to induce ASC differentiation into beige adipocytes in WAT for combating obesity. However, the poor solubility, poor bioavailability, and non-specific distribution of browning agents limit their application. Browning agents can be encapsulated in nanoparticles (NPs) to enhance their solubility and bioavailability. In this example, an ASC-targeting peptide, ASC2 as set forth in SEQ ID NO:2, was developed by using a combination of computational modeling, molecular docking, and adaptive peptide design algorithms. After coating NPs with the ASC2 peptide, the delivery of browning agents by the NPs to ASCs was increased. The delivery of browning agents to ASCs by NPs coated with the ASC2 peptide increased beige cell formation while decreasing body weight and fat mass. ASC-targeting peptide coated NPs can be administered via intravenous injection, subQ WAT injection, transdermal patches, or other administration routes. This targeted delivery approach for NPs enhances therapeutic agent delivery efficacy to ASCs while decreasing side effects and toxicity.

ASCs are multipotent progenitor cells found in the SVF of adipose tissue. ASCs play a critical role in adipose tissue maintenance, regeneration, and metabolic regulation. ASCs have significant therapeutic potential due to their accessibility, abundance, and ability to differentiate into various cell types. The accessibility, multipotency, and therapeutic versatility of ASCs makes them a valuable tool for regenerative medicine and targeted therapies, particularly in combating obesity and related metabolic disorders. In this example, the novel ASC-targeting peptide ASC2 as set forth in SEQ ID NO:2 was developed. The ASC-targeting peptide ligand ASC2 was also incorporated on the surface of NPs that can be engineered to carry and deliver a variety of agents to ASCs.

Trans-resveratrol (R) has the potential to induce ASCs in WAT to differentiate itno beige adipocytes for the purpose of treating obesity. However, the poor solubility, poor bioavailability, and non-specific distribution of trans-resveratrol (R) limit its application. It was demonstrated that the targeting peptide ASC1 (SEQ ID NO:1)-coated R-encapsulated nanoparticles (ASC1-R-NPs) can bind to the glycanation site-deficient decorin (ΔDCN) receptor, which is expressed on ASCs, and can deliver R into ASCs to induce browning of inguinal white adipose tissue (iWAT) in obese mice. Compared to ASC1-R-NPs, ASC2-coated R-encapsulated nanoparticles (ASC2-R-NPs) exhibit higher ASC-targeting specificity, enhanced R delivery to ASCs, increased beige cell formation, and exhibited greater accumulation in iWAT in C57BL/6J mice.

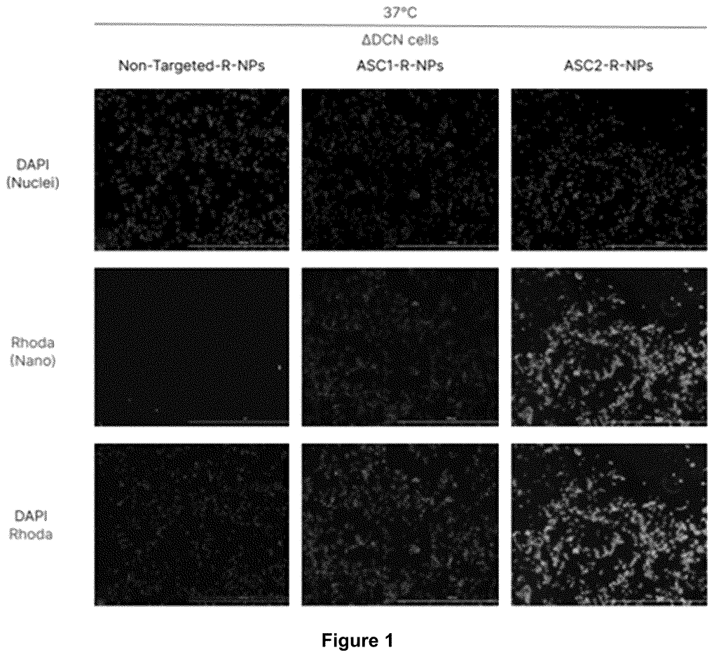

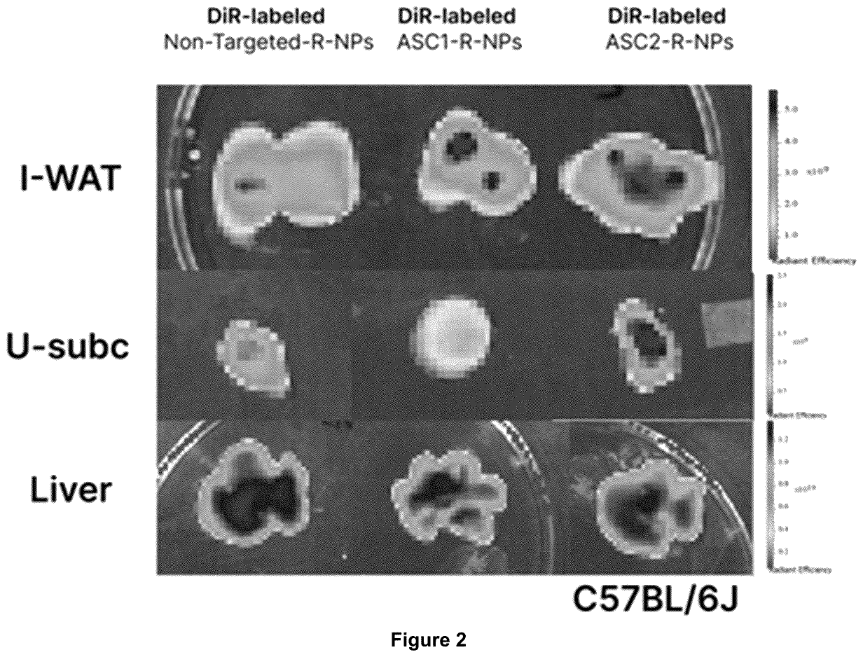

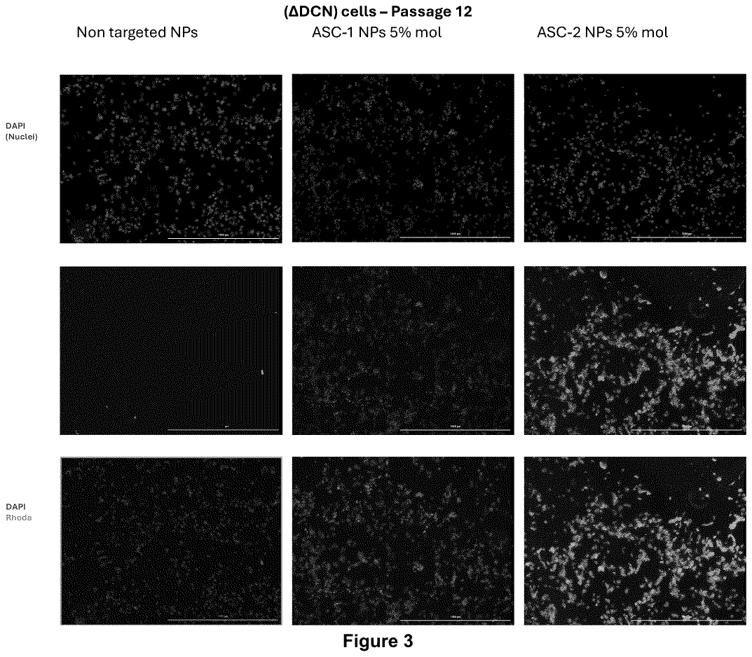

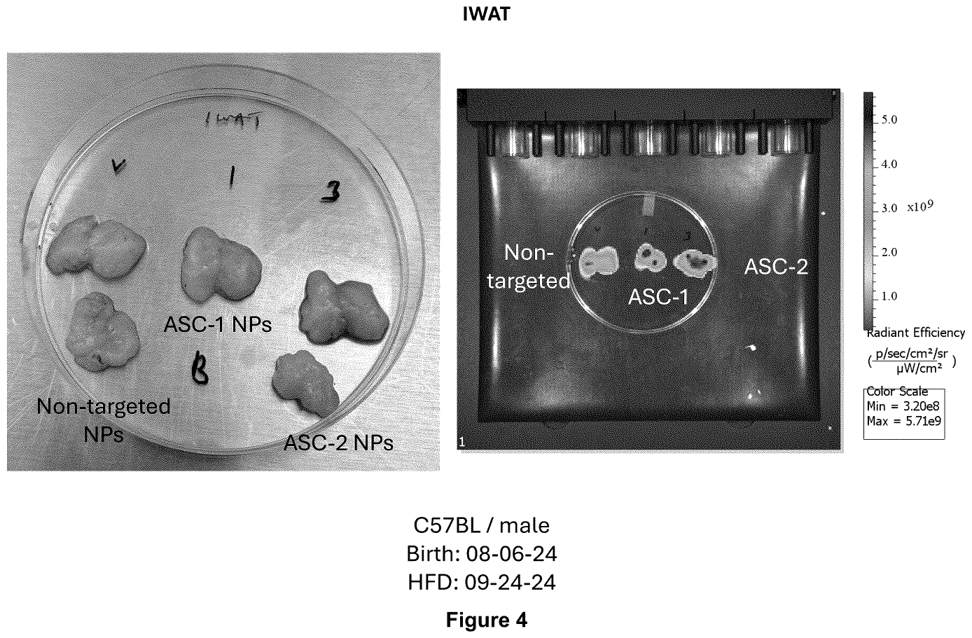

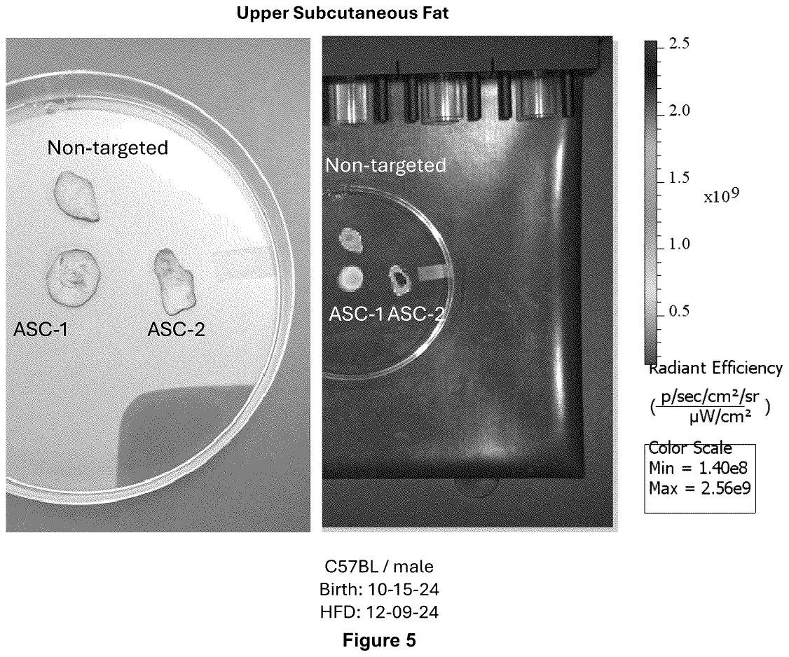

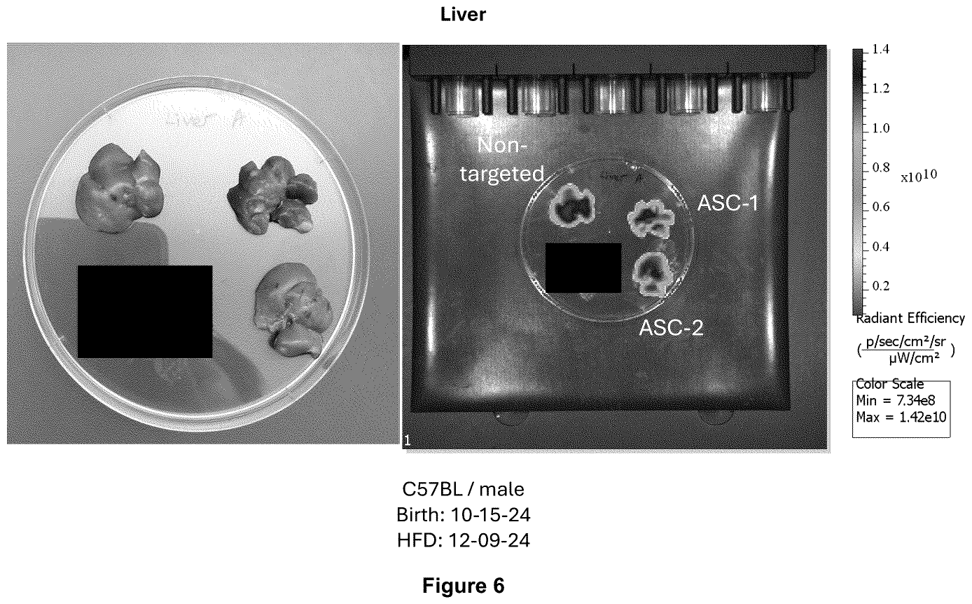

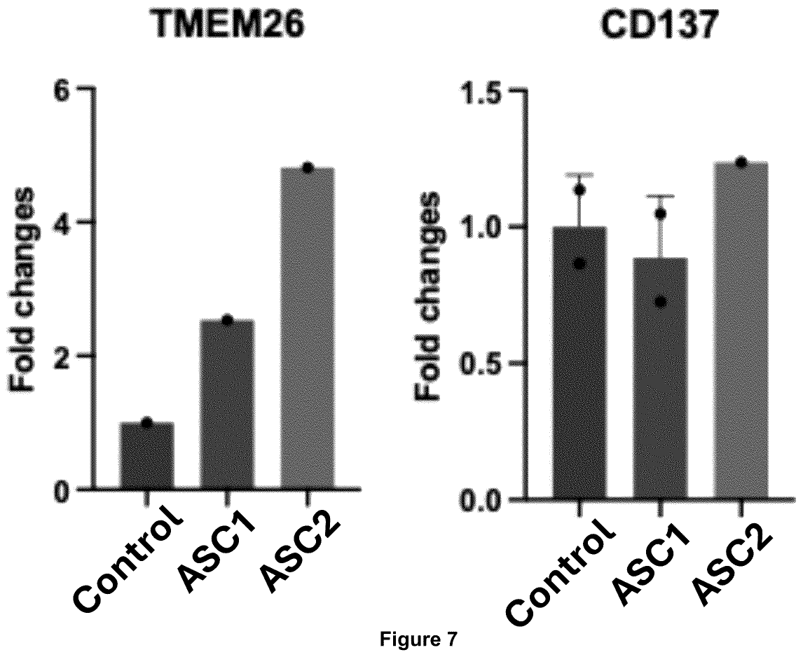

The mean particle size and (PDI) values were below 110 nm and 0.3, respectively, with R encapsulation efficiency of over 90%. Both ASC1-NPs and ASC2-NPs exhibited significantly enhanced binding and uptake in ΔDCN cells (FIG. 1 and FIG. 3). Both ASC1-R-NPs and ASC2-R-NPs increased cellular R content compared to non-targeted R-NPs, with ASC1-R-NPs causing a 1.6-fold increase and ASC2-R-NPs causing a 2.5-fold increase. Additionally, ASC1-R-NPs and ASC2-R-NPs increased Tmem26 expression by 1.8-fold and 4.8-fold, respectively. Additionally, ASX2-R-NPs increased CD137 expression by 1.3-fold. ASC2-NPs demonstrated significantly higher accumulation in iWAT and lower accumulation in the liver than ASC1-NPs in mice (FIG. 2 and FIG. 4 through FIG. 7).

Compared to ASC1-R-NPs, ASC2-R-NPs exhibited significantly higher target specificity to ASCs and subcutaneous WAT, resulting in increased cellular R delivery, beige adipocyte formation, and reduced off-target effects. The ASC2 peptide-based delivery is a breakthrough for combating obesity through browning WAT.

The targeted nanoparticles were shown to target and deliver a variety of agents to ASCs. Targeted nanoparticles can be used in the areas of preventing and treating obesity, diabetes, cardiovascular diseases, and other obesity-related metabolic diseases or disorders, preventing and treating types of cancer, including breast cancer, regenerative medicine, and reducing wrinkles.

The methods are described herein.

### Preparation of Nanoparticles

Non-targeted NPs were prepared using soy phosphatidylcholine (PC), α-tocopherol acetate, Kolliphor® HS15, and R through a sonication method. ASC1 and ASC2-targeted NPs were prepared by coating NPs with ASC1 or ASC2 peptides. NP size and polydispersity index were measured using Malvern Zetasizer. Fluorescent dyes were incorporated into non-targeted and ASC-targeted NPs, NP binding and uptake in ΔDCN-transduced 3T3-L1 cells (ΔDCN cells) were analyzed using EVOS® autofluorescence microscopy, while iWAT target specificity of intravenously administered NPs in mice was assessed using an in vivo imaging system (FIG. 1-3). ΔDCN cells were treated with non-targeted RNPs, ASC1-R-NPs, and ASC2-R-NPs at an R dose of 50 μM for 2 hours and cellular R content was quantified by extracting R with ethyl acetate and analyzing samples using HPLC.

### Rhodamine-Labeled Delivery

The fluorescence intensity of rhodamine-labeled Rnano and L-Rnano was equalized using a Agilent BioTek Cytation 5 absorbance microplate reader before treating cells to deliver an equivalent mass of nanoparticles. ΔDCN cells were cultured overnight prior to treatment. Cells were treated with rhodamine-labeled non-targeted-R-NPs, ASC1-R-NPs, and ASC2-R-NPs 37° C. for 2 hours. Cells were then washed three times with ice-cold 1×PBS and fixed with 3.7% formaldehyde, followed by washing with ice-cold 1×PBS three times. After staining nuclei with 4 ‘, 6-diamidine-2’-phenylindole dihydro chloride (DAPI), cells were mounted with Vectashield and visualized. Rhoda-labeled non-targeted-R-NPs, ASC1-R-NPs, and ASC2-R-NPs were imaged in orange (λ exc: 560 nm, λ em: 580 nm). Cell nuclei were stained by DAPI (2 exc: 461 nm). For equal comparison of all images, the exposure times were identical for all measurements for each fluorophore.

### In Vivo Delivery

Mice were intravenously injected with 1,1′-dioctadecyl-3,3,3′,3′-tetramethylindotricarbocyanine iodide (DiR) and rhodamine-labeled non-targeted-R-NPs, ASC1-R-NPs, and ASC2-R-NPs. Biodistribution and intensity of rhodamine-labeled NPs in mice and WAT deposits were visualized using an IVIS system. Higher DiR intensity indicates a higher level of nanoparticles.

The gene expression levels of browning markers in ΔDCN cells were measured using real time-PCR.

### Example 2: Adipose Stromal Stem Cell-Targeted Nanoparticle Formulations Testing

It was tested whether the inclusion of different surfactant species in Adipose Stromal Stem Cell-Targeted Nanoparticles resulted in differences in safety or efficacy. Nanoparticles were prepared similarly to the methods as described in Example 1. The surfactant Kolliphor® HS15 was replaced with Poloxamer 188 or Tween-80 for comparison studies.

Several poloxamer grades, including poloxamer 188, are FDA-approved as excipients and are widely used us emulsifiers or solubilizers in drug products, biologics, and other pharmaceutical formulations. The results of this Example suggest that nanoparticles comprising poloxamer 188 is safer than Kolliphor® HS15.

### MTT Cell Toxicity Assay

An MTT assay was performed for 72 hours using the 2 cell lines of ΔDCN Overexpressed 3T3-L1 cells and Bend.3 cell line. The results of the MTT assay indicate that nanoparticles comprising Poloxamer 188 did not exhibit significant cytotoxicity within the 0.005 mg/mL to 1 mg/mL range, whereas nanoparticles comprising Tween® 80 (polysorbate 80) showed gradual toxicity at higher concentrations, and nanoparticles comprising Kolliphor® HS15 demonstrated marked cytotoxicity at greater than or equal to 0.5 mg/mL. Therefore, Poloxamer 188 appears to be a primary candidate for local injections.

### Cell Binding Test

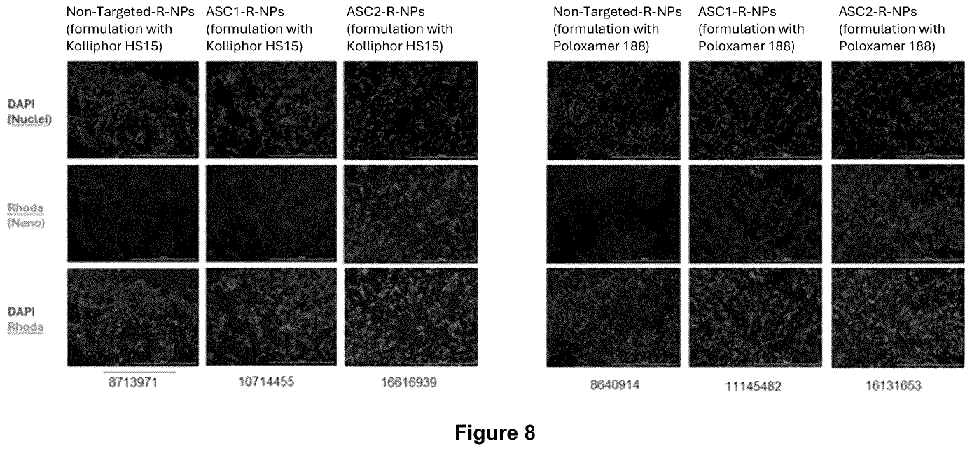

Results of a cell binding test showed that nanoparticles comprising Poloxamer 188 (P188) had a similar binding trend to nanoparticles comprising Kolliphor® HS15 (FIG. 8). The treatment duration of the cell binding test was 4 hours. ΔDCN cells were used in the cell binding test.

### Serum Biochemistry Profile for Toxicity Testing

Nanoparticles comprising P188 were administered to five mice over a 5-week period with biweekly local iWAT injections at a calculated drug dose. The 5-week nanoparticle administration period was started on 3 month old Male C57BL/6J mice. The mice were fed a high fat diet two weeks prior to the initiation of the nanoparticle administration. No toxicity concerns were observed based on serum biochemistry analyses (Table 2, below). Although glucose levels were slightly elevated, this is likely attributable to the use of obesity-induced C57BL/6J mice, in which glucose homeostasis is commonly impaired. Overall, the nanoparticle formulation appeared safe under the tested conditions.

TABLE 3
Serum Biochemistry Analysis

| | | Total | | | | | | |
|---|---|---|---|---|---|---|---|---|
| | Mice | Protein | Albumin | Globulin | ALP | Glucose | Total Bilirubin | Phosphorus |
| Group | ID | (g/dL) | (g/dL) | (g/dL) | (U/L) | (mg/dL) | (mg/dL) | (mg/dL) |
| ASC2-R-NPs | 31 | 4.60 | 2.00 | 2.60 | 62.00 | 173.00 | 0.10 | 5.70 |
| (formulation with | | | | | | | | |
| Poloxamer 188) | | | | | | | | |
| ASC2-R-NPs | 32 | 4.50 | 2.00 | 2.50 | 61.00 | 270.00 | 0.10 | 5.60 |
| (formulation with | | | | | | | | |
| Poloxamer 188) | | | | | | | | |
| ASC2-R-NPs | 33 | 4.40 | 2.00 | 2.40 | 53.00 | 177.00 | 0.20 | 5.70 |
| (formulation with | | | | | | | | |
| Poloxamer 188) | | | | | | | | |
| ASC2-R-NPs | 34 | 4.50 | 2.10 | 2.40 | 78.00 | 253.00 | 0.20 | 6.90 |
| (formulation with | | | | | | | | |
| Poloxamer 188) | | | | | | | | |
| ASC2-R-NPs | 35 | 4.60 | 2.00 | 2.60 | 55.00 | 142.00 | 0.20 | 6.40 |
| (formulation with | | | | | | | | |
| Poloxamer 188) | | | | | | | | |
| | Mean | 4.52 | 2.02 | 2.50 | 61.80 | 203.00 | 0.16 | 6.06 |
| | SD | 0.08 | 0.04 | 0.10 | 9.83 | 55.42 | 0.05 | 0.57 |
| Mouse Normal | | 3.5-7.2 | 2.5-3.0 | 1.0-4.2 | 35-96 | 62-175 | 0.0-0.9 | 5.7-9.2 |
| Range* | | | | | | | | |

| | Mice | Cholesterol | GGT | ALT | Calcium | Creatinine | BUN | Triglycerides | CPK |
|---|---|---|---|---|---|---|---|---|---|
| Group | ID | (mg/dL) | (U/L) | (U/L) | (mg/dL) | (mg/dL) | (mg/dL) | (mg/dL) | (U/L) |
| ASC2-R-NPs | 31 | 148.00 | 1** | 10.00 | 8.50 | 0.40 | 37.30 | 77.00 | 48.00 |
| (formulation with | | | | | | | | | |
| Poloxamer 188) | | | | | | | | | |
| ASC2-R-NPs | 32 | 132.00 | 1** | 18.00 | 8.60 | 0.20 | 32.70 | 109.00 | 32.00 |
| (formulation with | | | | | | | | | |
| Poloxamer 188) | | | | | | | | | |
| ASC2-R-NPs | 33 | 149.00 | 1** | 12.00 | 8.70 | 0.30 | 29.10 | 76.00 | 35.00 |
| (formulation with | | | | | | | | | |
| Poloxamer 188) | | | | | | | | | |
| ASC2-R-NPs | 34 | 155.00 | 3** | 14.00 | 8.40 | 0.20 | 30.30 | 93.00 | 28.00 |
| (formulation with | | | | | | | | | |
| Poloxamer 188) | | | | | | | | | |
| ASC2-R-NPs | 35 | 107.00 | 1** | 10.00 | 8.30 | 0.30 | 34.40 | 93.00 | ND |
| (formulation with | | | | | | | | | |
| Poloxamer 188) | | | | | | | | | |
| | Mean | 138.20 | | 12.80 | 18.50 | 0.28 | 32.76 | 89.60 | 35.75 |
| | SD | 19.41 | | 3.35 | 0.16 | 0.08 | 3.27 | 13.63 | 8.66 |
| Mouse Normal | | x | x | 17-77 | 7.1-10.1 | 0.2-0.9 | 8.0-33.0 | x | x |
| Range* | | | | | | | | | |

### In Vitro Flow Cytometry Analysis

Nanoparticle uptake was evaluated in ΔDCN-overexpressing cells following 24-hour incubation with saline, non-targeted R-NPs, ASC1-R-NPs, or ASC2-R-NPs at 5 μM and 10 μM. Rhodamine fluorescence-labeled R-NPs were used to quantify cellular uptake.

Saline-treated cells showed minimal background signal (0.53% positive). In contrast, all nanoparticle-treated groups demonstrated near-complete uptake: Across both concentrations, more than 98% of cells were nanoparticle-positive, indicating highly efficient internalization in ΔDCN-overexpressing cells independent of targeting ligand.

While uptake frequency was comparable across groups, differences emerged in intracellular accumulation (MFI): A clear ligand-dependent enhancement in fluorescence intensity was observed, with ASC2-R-NPs demonstrating the highest intracellular accumulation at both concentrations. The effect was more pronounced at 10 μM, indicating a dose-dependent amplification of targeted nanoparticle uptake.

Although ΔDCN-overexpressing cells exhibit near-universal nanoparticle uptake regardless of formulation, targeted nanoparticles (particularly ASC2-R-NPs) significantly increase intracellular accumulation, as reflected by elevated MFI values. These findings support enhanced binding and/or internalization efficiency mediated by the ASC-targeting ligand in the ΔDCN-overexpressing model.

### In Vivo Flow Cytometry Analysis of Nanoparticle Uptake in Adipose Stromal Cell (ASC) Populations

Stromal vascular fraction (SVF) was isolated from inguinal white adipose tissue (iWAT) and subsequently processed for flow cytometric analysis. Briefly, adipose tissue was enzymatically digested, filtered to remove debris, and centrifuged to obtain the SVF pellet. Cells were then stained with antibodies.

Flow cytometric analysis was performed following sequential gating on CD45⁻/CD31⁻ cells to exclude hematopoietic and endothelial populations, followed by identification of the CD34⁺/CD29⁺ adipose stromal cell (ASC) fraction. DiD fluorescence was used to quantify nanoparticle (R-NP) uptake.

Targeted R-NP formulations (ASC1⁻R-NP and ASC2⁻R-NP) maintained high uptake efficiency within the CD45⁻/CD31⁻/CD34⁺/CD29⁺ ASC population. ASC2⁻R-NPs demonstrated the strongest intracellular accumulation based on MFI, indicating superior targeting performance compared to non-targeted RNP.

While overall uptake frequency (DiD⁺%) was comparable among groups, ASC2⁻R-NPs exhibited the highest fluorescence intensity, suggesting enhanced nanoparticle internalization per cell rather than an increase in the proportion of positive cells.

Male C57BL/6J were used the In Vivo Flow Cytometry Analysis experiments.

### Example 3: Sequences

ASC1 Peptide
(SEQ ID NO: 1)
GSWKYWFGEGGC
ASC2 Peptide 1
(SEQ ID NO: 2)
WRFWLKRGGC
ASC2 Peptide 2
(SEQ ID NO: 3)
WRFWLKR
ASC2 Peptide 3
(SEQ ID NO: 4)
WRFWLKRC
ASC2 Peptide 4
(SEQ ID NO: 5)
CGGWRFWLKR
ASC2 Peptide 5
(SEQ ID NO: 6)
CWRFWLKR

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising at least one targeting peptide comprising an amino acid sequence least 90% identical to SEQ ID NO:2, SEQ ID NO:3, SEQ ID NO:4, SEQ ID NO:5, or SEQ ID NO:6.

2. The composition of claim 1, wherein the composition comprises a nanoparticle comprising the at least one targeting peptide on or conjugated to at least a portion of the surface of the nanoparticle.

3. The composition of claim 1, further comprising a therapeutic agent.

4. The composition of claim 3, wherein the therapeutic agent comprises an adipocyte stromal cell browning agent.

5. The composition of claim 3, wherein the therapeutic agent comprises at least one of the group consisting of: resveratrol, trans-resveratrol, and quercetin.

6. The composition of claim 3, wherein the composition comprises the at least one targeting peptide conjugated to the therapeutic agent directly or indirectly through a linker.

7. The composition of claim 3, wherein the therapeutic agent is encapsulated within, adhered to a surface of, or integrated into the structure of a nanoparticle.

8. The composition of claim 1, wherein the composition increases bioavailability of resveratrol, or a derivative, prodrug, or pharmaceutically acceptable salt thereof, trans-resveratrol, or a derivative, prodrug, or pharmaceutically acceptable salt thereof, quercetin, or a derivative, prodrug, or pharmaceutically acceptable salt thereof, or any combination thereof.

9. The composition of claim 1, wherein the composition induces browning of a white adipose tissue.

10. The composition of claim 1, wherein the composition induces the differentiation of an adipose stromal cell.

11. The composition of claim 10, wherein the differentiation comprises differentiation to a brown adipocyte or a beige adipocyte.

12. The composition of claim 1, wherein the composition is selected from the group consisting of: an oral formulation, an injectable solution, a solution for transdermal delivery, an injectable hydrogel, a surgically implanted hydrogel, and any combination thereof.

13. The composition of claim 2, wherein the nanoparticle comprises a poloxamer.

14. The composition of claim 13, wherein the surfactant comprises poloxamer 188.

15. A method of treating a subject comprising the step of:
administering the composition of claim 1.

16. The method of claim 15, wherein the subject has a metabolic disease, a metabolic disorder, or cancer.

17. The method of claim 15, wherein the subject has breast cancer.

18. The method of claim 15, wherein the treatment induces weight loss, reduces wrinkles, or promotes tissue regeneration.

19. The method of claim 15, wherein the subject has a metabolic disease or disorder selected from the group consisting of: obesity, obesity-related disease or disorder, diabetes, fatty liver disease, non-alcoholic fatty liver disease, cardiovascular disease, heart disease, stroke, hypertension, idiopathic intracranial hypertension, coronary artery disease, atrial fibrillation, atherosclerosis, venous thromboembolism, obstructive sleep apnea, obesity hypoventilation syndrome, metabolic syndrome, insulin resistance, esophageal adenocarcinoma, gastric cancer, colorectal cancer, hepatocellular carcinoma, cholangiocarcinoma, pancreatic cancer, endometrial carcinoma, ovarian cancer, breast cancer, renal cell carcinoma, multiple myeloma, and any combination thereof.

20. The method of claim 15, wherein the composition is administered via oral administration, injection, or a combination thereof.
